(19) United States
(12) Patent Application Publication
(10) Pub. No.: US 2026/0188515 A1
(43) Pub. Date: Jul. 2, 2026

(54) SYSTEM AND METHOD FOR NORMALIZING MULTI-SOURCE TIME-SERIES DATA USING ADAPTIVE COHORT-BASED HANDICAP MODELING

(71) Applicant: WellDuel, Dover, DE (US)

(72) Inventor: James McGovern, North Kingstown, RI (US)

(73) Assignee: WellDuel, Dover, DE (US)

(21) Appl. No.: 19/436,194

(22) Filed: Dec. 30, 2025

Related U.S. Application Data
(60) Provisional application No. 63/740,924, filed on Dec. 31, 2024.

(51) Int. Cl. G16H 50/30; G06N 3/088; G16H 10/60; G16H 50/70

(52) U.S. Cl. CPC G16H 50/30; G06N 3/088; G16H 10/60; G16H 50/70

(57) ABSTRACT

A computing system for generating handicap-adjusted health scores processes multi-source health metric data collected from heterogeneous devices and activity modalities. The system normalizes and calibrates unstructured health data using schema-conformant transformation logic and device-specific correction parameters. User-specific baseline values are computed over configurable rolling windows and compared to cohort-level reference parameters derived from demographic and device-based population segmentation. Deviation values are corrected using demographic and device normalization models to generate structured adjustment vectors. The system applies context-aware scoring logic to compute normalized health scores that reflect individual performance patterns relative to statistically similar user cohorts. Scoring outputs are configured for competitive presentation via a user interface and may support leaderboard ranking, team-based scoring, or context-specific evaluations across diverse activity types.

### CROSS-REFERENCE TO RELATED APPLICATIONS

This application claims priority to U.S. Provisional Patent Application No. 63/740,924 , entitled “Balanced Health Index (BHI): A Gamified System for Fair Competition in Health Tracking,” filed on Dec. 31, 2024, the entire disclosure of which is incorporated herein by reference in its entirety.

### FIELD OF THE ART

The systems and methods disclosed herein relate generally to computing systems for time-series data processing and, more specifically, to systems for normalizing heterogeneous metric data from multiple sources and generating cohort-adjusted performance scores using adaptive modeling techniques.

### BACKGROUND

### Discussion of the State of the Art

Computing systems increasingly ingest metric data streams from heterogeneous health tracking devices, including wearable sensors, smartphone-based applications, and third-party health platforms. These data streams exhibit structural and semantic inconsistencies resulting from differences in sampling rates, measurement units, schema definitions, and sensor calibration methods. As a result, metric data representations may vary significantly between users, even for nominally identical activities.

Existing systems that process health metric data often perform absolute comparisons of raw values, such as step counts, resting heart rate, or calories burned. These absolute comparisons fail to account for inter-user variability in physiological baselines, device-specific measurement characteristics, and demographic influences. Consequently, score-based competition systems, gamification platforms, and health tracking leaderboards produce skewed or inequitable rankings, particularly when aggregating data across users with different device types, age groups, health conditions, or activity modalities.

Conventional approaches to normalizing health performance data frequently apply fixed formulaic adjustments or pre-defined demographic buckets. These methods lack the adaptability and technical sophistication required to perform real-time, user-specific normalization using machine-executed processing. Moreover, current systems do not incorporate rolling baseline models, population cohort segmentation, or dynamic calibration factors derived from device metadata or behavioral context.

Some platforms attempt to address performance normalization by computing user-specific progress over time or comparing scores within restricted demographic segments. However, these approaches are typically heuristic-based, operate within a single device ecosystem, and fail to support cross-platform normalization or adaptive scoring logic across heterogeneous user populations.

Existing systems lack the technical infrastructure to perform schema-conformant normalization across diverse health tracking sources in a scalable and schema-consistent manner. Current approaches do not dynamically adjust for device-specific measurement variance, nor do they segment users into adaptive peer groups using demographic or behavioral context. Furthermore, existing scoring mechanisms fail to account for inter-user variability in baseline health metrics, resulting in performance assessments that do not reflect relative effort or improvement.

### SUMMARY

Systems and methods in accordance with various embodiments of the present disclosure address limitations in conventional computing approaches to generating equitable health scores across heterogeneous users and devices. In particular, embodiments described herein normalize unstructured metric data from diverse health tracking sources, calibrate values using device-specific profiles, segment users into adaptive population cohorts, compute deviation-based adjustment factors, and generate handicap-adjusted scores suitable for cross-user comparison within a competitive health platform.

For example, unstructured health metric data is obtained from a plurality of health tracking sources. The data includes time-series values collected across heterogeneous devices and activity modalities, including step count, heart rate, sleep duration, or perceived exertion. The raw data varies in format, sampling interval, and measurement fidelity depending on the originating device or platform.

The heterogeneous metric data is normalized to produce schema-aligned representations. Normalization includes converting measurement units, aligning temporal sampling rates, and mapping source-specific fields to a unified data schema. These operations enable consistent downstream analysis regardless of device origin or data structure.

Standardized metric values are calibrated using device profile data. The device profile data includes known measurement deviations, accuracy parameters, or empirical bias coefficients associated with each supported device. Calibration adjusts the normalized values to reflect corrected estimates of the user's actual physiological state.

For each health metric, a rolling baseline is generated for the user using the calibrated values. The baseline represents the user's typical recent performance and is computed over a configurable time interval using time-aware aggregation techniques such as weighted averaging or exponential decay.

The user population is segmented into adaptive cohorts based on demographic data and device metadata. Segmentation may be performed using clustering algorithms or classification logic that groups users by similar characteristics, such as age, device type, or measurement behavior. Each cohort defines a reference group for relative performance comparison.

The user is assigned to a selected population cohort, and corresponding cohort reference parameters are retrieved. These parameters represent typical or expected values for each health metric within the assigned cohort and provide the basis for evaluating relative deviation.

Deviation values are computed by comparing the user's baseline to the cohort reference parameters. These deviations are then modified by correction factors derived from user-specific attributes, including demographic, behavioral, device-related, or mental health considerations.

A handicap adjustment vector is generated from the corrected deviation values. Each element in the vector represents a per-metric adjustment applied to current metric values, resulting in a normalized score that reflects relative user performance within the cohort-adjusted scoring framework.

The normalized score is output for presentation via a user interface associated with a health competition platform. In certain embodiments, scores may support leaderboard generation, token-based rewards, or achievement tracking. Model retraining may be triggered based on drift detection in cohort parameters or population composition over time. When retraining is initiated, normalized training data is supplied to model training system 114, which applies population-level learning logic to generate updated cohort segmentation and handicap adjustment models. These trained models are then deployed to model-based inference system 110 for active scoring workflows.

The disclosed systems and methods provide a computer-implemented solution to a technical problem involving inconsistent and inequitable scoring of heterogeneous health metric data across disparate device ecosystems. The invention transforms raw, unstructured time-series health data into calibrated, schema-conformant representations that enable structured population cohort modeling, per-user normalization, and real-time handicap-adjusted scoring. The system operates across multiple device types and sampling schemas, applies rule-based and machine-learned normalization logic, and produces output scores that reflect both user-specific baselines and population-wide health trends. These operations are performed by machine-executed processing components and are not achievable through manual or purely mental activity. In particular, model training system 114 enables the dynamic construction of scoring models tailored to evolving user distributions and device behaviors, ensuring fairness, consistency, and adaptability at scale. The disclosed architecture supports large-scale, privacy-compliant deployment environments and facilitates integration into technical applications involving mobile health tracking, wellness gamification, and adaptive feedback systems.

Various other functions and advantages are described and suggested below as may be provided in accordance with the various embodiments.

### BRIEF DESCRIPTION OF THE DRAWINGS

The accompanying drawings illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1 illustrates an example system architecture for health-based scoring using cohort modeling and adjustment logic in accordance with various embodiments.

FIG. 2 illustrates example components of the competition management system in accordance with various embodiments.

FIG. 3 illustrates example components of the model-based inference system in accordance with various embodiments.

FIG. 4 illustrates an example training pipeline for cohort modeling and adjustment factor generation in accordance with various embodiments.

FIG. 5 illustrates an example process for model training and drift detection in accordance with various embodiments.

FIG. 6 illustrates an example process for computing a normalized score in accordance with various embodiments.

FIG. 7 illustrates an example process for generating adjustment vectors in accordance with various embodiments.

FIG. 8 illustrates an example device-level architecture that can support various embodiments.

FIG. 9 illustrates example components of a computing device in accordance with various embodiments.

FIG. 10 illustrates an example architecture of a system in accordance with various embodiments.

FIG. 11 illustrates example components of a computing device in accordance with various embodiments.

### DETAILED DESCRIPTION

The embodiments described herein relate to systems and methods for generating normalized health scores through a layered modeling architecture that accounts for individualized performance patterns, cohort-relative deviation, and demographic or device-specific correction. The system is operable to ingest heterogeneous biomarker and activity data from multiple sources, normalize and calibrate time-series values, segment users into statistically similar population cohorts, and compute structured adjustment vectors representing individualized deviation from cohort reference norms. In various embodiments, the system includes components for schema-conformant data ingestion, feature vector calibration, cohort modeling, baseline computation, adjustment vector generation, and scoring logic application. The system applies machine-executed transformations to unstructured or semi-structured health data collected from diverse tracking platforms, producing temporally aligned, calibrated metric vectors and per-user scores suitable for competitive ranking, feedback delivery, or multi-context evaluation. In certain implementations, the system supports retraining workflows for cohort models, version-controlled adjustment parameters, and configuration-driven scoring logic to support fairness, reproducibility, and cross-platform deployment.

In certain embodiments, the disclosed system generates comparative scores by accounting for differences in individual physiology, baseline performance, and device variability —even when users complete the same type of activity. For example, when two users perform a treadmill workout, the system does not rely solely on raw metrics such as speed, distance, or heart rate. Instead, it analyzes a set of biomarkers—such as resting heart rate, sleep quality, and blood glucose variability—relative to each user's recent historical trends. It then adjusts these values by comparing the user's typical patterns to a larger reference population that shares similar demographic traits and device characteristics. The system may also apply statistical correction models to account for age, sex, and known sensor differences between devices. This layered approach produces a normalized score that reflects not just how much was done, but how that effort compares to what is typical for the individual and expected from peers with similar profiles. This method can also be extended to compare users across different types of activity or environmental contexts, while still maintaining fairness and interpretability.

Although described in connection with health gamification and competition environments, the disclosed systems and methods may be applied in other technical domains that require individualized normalization of time-series metric data across heterogeneous data sources. For example, applications may include personalized training systems, recovery monitoring platforms, occupational safety dashboards, or mental wellness tracking systems. Each of these domains benefits from schema-conformant data alignment, cohort-based comparison models, and real-time scoring logic applied to multi-modal user data under device and demographic constraints.

### Conceptual Architecture

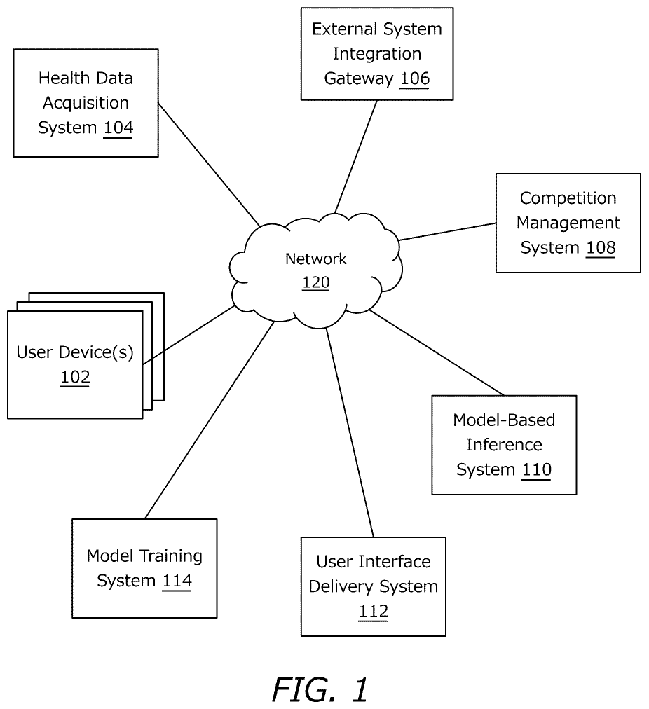

FIG. 1 illustrates an example system architecture for health-based scoring using cohort modeling and adjustment logic in accordance with various embodiments. It should be understood that reference numbers are carried over between figures for similar components for purposes of simplicity of explanation, but such usage should not be construed as a limitation on the various embodiments unless otherwise stated. As shown, FIG. 1 includes user device(s) 102, health data acquisition system 104, external system integration gateway 106, competition management system 108, model-based inference system 110, user interface delivery system 112, model training system 114, and a network 120 over which the various components communicate and exchange data.

The various components described herein are exemplary and may be implemented in combination, subdivision, or consolidation across one or more computing devices or servers, as would be understood by a person of ordinary skill in the art, without departing from the scope of the invention. The various components described herein are exemplary and for illustration purposes only and any combination or subcombination of the various components may be used as would be apparent to one of ordinary skill in the art. The system may be reorganized or consolidated, as understood by a person of ordinary skill in the art, to perform the same tasks on one or more other servers or computing devices without departing from the scope of the invention.

User device(s) 102 comprise one or more network-connected computing devices operable to transmit multi-source health metric data and render interface components for interacting with normalized scores, handicap adjustments, and cohort-based feedback workflows. User device(s) 102 may include smartphones, tablets, wearable trackers, connected fitness equipment (e.g., smart bikes, rowing machines), or other devices configured to execute native or browser-based applications that communicate with the systems described herein. Through these interfaces, users may contribute time-series activity data, receive calibrated score feedback, and interact with ongoing competition formats. In certain embodiments, user device(s) 102 transmit device-generated health data to the health data acquisition system 104 and receive output visualizations from the user interface delivery system 112.

User device(s) 102 include, generally, a computer or computing device including functionality for communicating (e.g., remotely) over a network 120. Data may be collected from user device(s) 102, and data requests may be initiated from each user device. User device(s) 102 may be a server, a desktop computer, a laptop computer, personal digital assistant (PDA), a smart phone or other cellular or mobile phone, wearable sensor, or mobile gaming device, among other suitable computing devices. User device(s) 102 may execute one or more applications, such as a web browser (e.g., Microsoft Windows Internet Explorer, Mozilla Firefox, Apple Safari, Google Chrome, and Opera, etc.), or a dedicated application to submit user data or respond to system prompts over network 120.

In particular embodiments, each user device may be an electronic device including hardware, software, or embedded logic components or a combination of two or more such components and capable of carrying out the appropriate functions implemented or supported by the user device. For example and without limitation, a user device may be a desktop computer system, a notebook computer system, a handheld electronic device, or a wearable fitness tracker. The present disclosure contemplates any user device. A user device may enable a network user at the user device to access network 120. A user device may enable its user to communicate with other users at other user devices.

A user device may have a web browser, such as MICROSOFT INTERNET EXPLORER, GOOGLE CHROME or MOZILLA FIREFOX, and may have one or more add-ons, plug-ins, or other extensions. A user device may enable a user to enter a Uniform Resource Locator (URL) or other address directing the web browser to a server, and the web browser may generate a Hyper Text Transfer Protocol (HTTP) request and communicate the HTTP request to a server. The server may accept the HTTP request and communicate to the user device(s) 102 one or more Hyper Text Markup Language (HTML) files responsive to the HTTP request. The user device(s) 102 may render a web page based on the HTML files from server for presentation to the user. The present disclosure contemplates any suitable web page files. As an example and not by way of limitation, web pages may render from HTML files, Extensible Hyper Text Markup Language (XHTML) files, or Extensible Markup Language (XML) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a web page encompasses one or more corresponding web page files (which a browser may use to render the web page) and vice versa, where appropriate.

The user device(s) 102 may also include a native or web-based application that is loaded onto the user device(s) 102. The application may acquire health-related metric data from device sensors, communicate with backend processing systems, and display real-time score feedback within the application interface.

Exemplary user devices are illustrated in some of the subsequent figures provided herein. This disclosure contemplates any suitable number of user devices, including computing systems taking any suitable physical form. As example and not by way of limitation, computing systems may be an embedded computer system, a system-on-chip (SOC), a single-board computer system (SBC) (such as, for example, a computer-on-module (COM) or system-on-module (SOM)), a desktop computer system, a wearable activity tracker, an interactive kiosk, a mobile telephone, a tablet, a server, or a combination of two or more of these. Where appropriate, the computing system may include one or more computer systems; be unitary or distributed; span multiple locations; span multiple machines; or reside in a cloud, which may include one or more cloud components in one or more networks. Where appropriate, one or more computing systems may perform without substantial spatial or temporal limitation one or more steps of one or more methods described or illustrated herein. As an example, and not by way of limitation, one or more computing systems may perform in real time or in batch mode one or more steps of one or more methods described or illustrated herein. One or more computing systems may perform at different times or at different locations one or more steps of one or more methods described or illustrated herein, where appropriate.

Health data acquisition system 104 is operable to ingest multi-source health data from a plurality of health tracking sources. More specifically, health data acquisition system 104 receives unstructured health metric data comprising time-series values collected from diverse user device types, including wearables, smartphones, fitness equipment, biometric sensors, and third-party health tracking platforms. The unstructured health metric data includes heterogeneous metric values such as heart rate, heart rate variability, step count, calories burned, distance, sleep duration, and perceived exertion, each of which may vary in format, sampling frequency, measurement unit, and schema definition depending on the originating device or data source.

In certain embodiments, health data acquisition system 104 includes one or more transport layer modules configured to establish authenticated communication sessions with device-specific APIs, SDKs, or platform interfaces. For example, health data acquisition system 104 may include authorization adapters that support OAuth 2.0 or token-based access protocols to collect metric data from commercial platforms such as Apple HealthKit, Google Fit, Garmin Connect, or Fitbit Web APIs. In other embodiments, health data acquisition system 104 may receive raw device output files (e.g., JSON, CSV, binary logs) via encrypted upload sessions initiated by edge devices configured to collect metrics locally.

Health data acquisition system 104 performs format validation, schema inference, and temporal alignment for incoming metric data records. For example, upon receiving device payloads, the system parses metric records to extract timestamped observations, determines the originating schema version, and assigns source tags for device lineage tracking. The extracted data is passed to normalization module(s) via a structured handoff protocol that includes source identifiers, confidence metadata, and time-series integrity indicators. In certain embodiments, health data acquisition system 104 also flags anomalous payloads for exclusion, including records that fall outside expected physiological ranges or those identified as duplicates through hash-based collision detection.

In embodiments where user consent or regulatory compliance applies, health data acquisition system 104 may interface with a permissions management layer that validates user-level authorization and logs data access events for auditability. Data streams processed by health data acquisition system 104 are stored in raw data storage system 208 and made available for subsequent normalization, calibration, and baseline generation as described in later components of competition management system 108.

External system integration gateway 106 is operable to receive contextual data streams and metadata from third-party platforms relevant to health metric calibration, demographic segmentation, and cohort normalization. More specifically, external system integration gateway 106 establishes authenticated communication links with external data providers, including but not limited to electronic health record (EHR) systems, insurance wellness platforms, demographic datasets, device certification repositories, and environmental context services. These integrations enable the computing system to supplement raw health metric data with additional attributes required for user cohort assignment and personalized scoring adjustments.

In various embodiments, external system integration gateway 106 comprises a set of modular interface adapters configured to support both push and pull data exchange protocols. For example, external system integration gateway 106 may include RESTful API connectors for retrieving user age, gender, height, or known health conditions from a provider-linked wellness portal, or for importing device metadata—such as model version, firmware level, or sensor calibration specifications—from a manufacturer's certification database. In some embodiments, the integration gateway uses asynchronous webhooks or batch data import routines to process large-scale demographic updates or population-wide environmental data (e.g., air quality, elevation, humidity) that may influence scoring behavior.

Upon receipt of third-party data, external system integration gateway 106 performs schema alignment, source validation, and field-level mapping to ensure compatibility with the data models used by competition management system 108 and model-based inference system 110. For example, demographic values may be mapped to a normalized internal format using a lookup table or rule-based parser, while device metadata may be validated against known device profile entries stored in a calibration datastore. The gateway further includes timestamp reconciliation logic to synchronize context data with the corresponding time intervals associated with user health metrics.

In certain embodiments, external system integration gateway 106 operates within a secure microservices container and includes audit logging functionality to record data access events, schema transformation history, and authorization state. The output of external system integration gateway 106 is routed to cohort assignment module 218, calibration module 216, and other components of competition management system 108 for structured incorporation into the handicap modeling workflow.

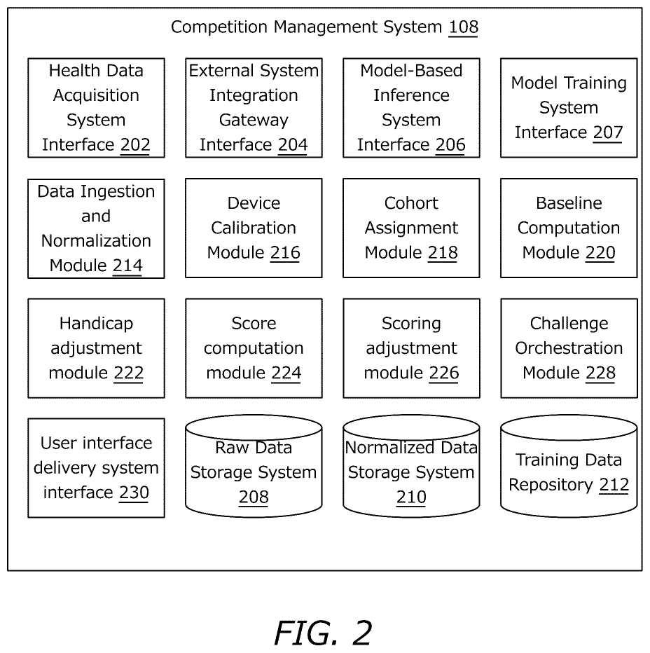

Competition management system 108 is operable to coordinate health competition logic, compute normalized health scores, and manage user participation across a competitive scoring platform. More specifically, competition management system 108 orchestrates the processing of multi-source health data, including normalization, calibration, cohort assignment, baseline modeling, scoring computation, and scoring adjustment operations. In certain embodiments, competition management system 108 receives health metric data from health data acquisition system 104 and external system integration gateway 106, transforms the data into calibrated representations, applies cohort-based handicap adjustments, and transmits resulting scores to user interface delivery system 112 for presentation. Competition management system 108 is further described in FIG. 2.

In various embodiments, competition management system 108 performs schema normalization to reconcile heterogeneous health metric values across disparate devices and sampling modalities. For example, step count, heart rate, and sleep metrics received in inconsistent formats may be transformed into schema-aligned vectors using unit conversion and field mapping logic. Competition management system 108 further applies device calibration coefficients derived from device profile data, adjusting raw values based on known measurement deviations or fidelity parameters. Using the calibrated metrics, the system computes a rolling baseline for each user based on a configurable time window, such as a 7-day or 14-day interval. The computed baseline is compared to one or more cohort reference parameters retrieved based on demographic or behavioral segmentation.

Deviation values are generated for each metric based on the difference between the user-specific baseline and the assigned cohort reference value. These deviations may be adjusted using correction factors such as demographic coefficients, device metadata, or behavioral context indicators. Competition management system 108 generates a handicap adjustment vector using the corrected deviations and applies this vector to the user's current metrics to produce a normalized score. In certain embodiments, the normalized score is a composite value computed from multiple weighted per-metric adjustments, each reflecting relative performance against a dynamically modeled peer group.

Competition management system 108 transmits normalized scores and related scoring metadata to user interface delivery system 112 for leaderboard rendering, achievement tracking, or token-based reward generation. The system may also interface with model-based inference system 110 to retrieve trained scoring models, trigger retraining workflows in response to drift, or log scoring events for audit purposes. In certain embodiments, competition management system 108 supports configurable scoring logic, including different competition formats, scoring weights, and evaluation periods, enabling flexible deployment across individual, team-based, or organization-wide wellness programs.

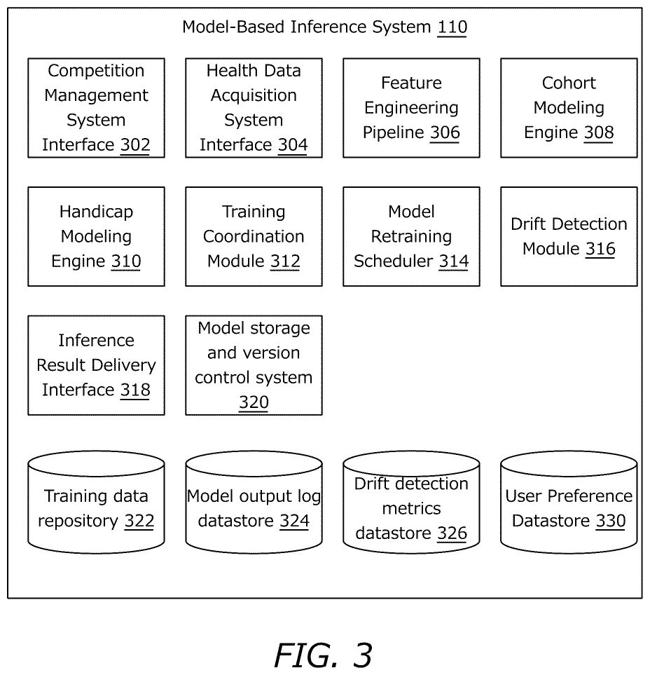

Model-based inference system 110 is operable to generate, apply, and manage predictive models for population cohort segmentation and handicap-adjusted scoring. More specifically, model-based inference system 110 receives normalized and calibrated health metric data, processes the data using structured feature engineering and model inference logic, and outputs scoring adjustments, cohort classifications, or retraining triggers in response to evolving user data or population distributions. Model-based inference system 110 is further described with respect to its internal components and supporting modules in FIG. 3.

In various embodiments, model-based inference system 110 interfaces with competition management system 108 to retrieve user-specific metric vectors and demographic attributes. The system applies a trained cohort modeling engine to determine appropriate population assignments based on similarity metrics, clustering thresholds, or classification confidence scores. The output of this modeling process informs the selection of cohort reference parameters used during deviation computation and scoring normalization.

Model-based inference system 110 may also apply trained handicap modeling logic to generate per-metric adjustment values reflecting cohort-relative performance. For example, the system may compute predicted adjustment values based on feature vectors that incorporate rolling user baselines, demographic coefficients, device calibration metadata, and behavioral attributes. These values are returned to competition management system 108 as structured adjustment vectors used during score generation.

In certain embodiments, model-based inference system 110 supports scheduled or event-triggered model retraining based on scoring stability indicators or population drift metrics. The system monitors deviation consistency, cohort accuracy thresholds, and fairness distribution to determine when retraining is warranted. Upon detecting drift, the system may coordinate with centralized data repositories to retrieve updated training records, execute retraining tasks using historical and current data, validate model outputs against predefined performance metrics, and publish new model versions for operational use. The trained models used during inference are generated and updated by model training system 114, which applies cohort-based learning logic to create population-specific scoring models.

Model-based inference system 110 may maintain model state information, version control metadata, and evaluation statistics for each deployed model. The system enables reproducibility of model outputs by logging input vectors, model weights, and scoring results for each inference cycle. In some embodiments, the system supports A/B testing of alternate scoring models or cross-validation against holdout datasets to assess generalization and mitigate scoring bias across demographic groups.

User interface delivery system 112 is operable to generate, manage, and transmit interactive graphical user interfaces for presenting health metric scores, competition progress, and user-specific feedback. More specifically, user interface delivery system 112 receives normalized scores, adjustment vectors, and cohort-related context from competition management system 108 and model-based inference system 110, formats these values for display, and renders outputs via client-facing components on user device(s) 102. The system supports both web-based and native application delivery environments and is configured to accommodate platform-specific interface protocols and resolution constraints.

In various embodiments, user interface delivery system 112 is operable to provide users with personalized dashboards that include time-series visualizations of health metric trends, normalized score history, cohort-relative performance indicators, and status within active competitions. The system may display individual and team leaderboards, percentile rankings, adjustment factor breakdowns, and token accumulation metrics where applicable. To ensure timely updates, user interface delivery system 112 may support server push architectures (e.g., WebSockets, Server-Sent Events) or periodic polling intervals for retrieving score updates and notification payloads.

In certain embodiments, user interface delivery system 112 supports interactive elements for challenge enrollment, goal selection, and review of historical performance data. For example, the interface may allow a user to select from various competition formats (e.g., head-to-head, group, bracket), review their cohort assignment history, or inspect the impact of calibration and demographic factors on recent scores. The system may further expose trend analysis visualizations and behavioral insights derived from longitudinal health metric tracking, enabling users to understand their performance relative to evolving baselines or population shifts.

User interface delivery system 112 is operable to generate, transmit, and render interface components for presenting handicap-adjusted health scores and facilitating user interaction within a competition platform. More specifically, user interface delivery system 112 is configured to deliver visualizations of metric scores, cohort-adjusted performance rankings, and real-time feedback via browser-based or native mobile applications. The user interface delivery system 112 may generate dashboards, charts, progress trackers, and achievement indicators using structured outputs received from model-based inference system 110. The presentation format may adapt to user context, device type, or competition format, and may include configurable display modes for team-based views, historical trends, or goal tracking.

In certain embodiments, user interface delivery system 112 is further operable to receive user-generated input for competition enrollment, device linking, privacy settings, and personal health goals. The received input may include demographic information (e.g., age, gender), user preferences (e.g., preferred metric weighting or challenge types), and device configuration selections. The system may transmit this user-provided information to competition management system 108 for processing, storage, and cohort assignment, or to model-based inference system 110 for dynamic score recalibration. In addition, user interface delivery system 112 may support consent capture for health data usage, provide notification settings, and receive manual feedback that influences model retraining workflows. In some embodiments, user interface delivery system 112 includes device-specific rendering logic that optimizes data display for smartphones, tablets, wearable screens, or embedded fitness equipment interfaces.

User interface delivery system 112 includes access control logic to support authentication and authorization workflows, ensuring that competition data, health metrics, and adjustment parameters are only viewable by permitted users. The system may integrate with external identity providers or health data platforms to support federated login and privacy-compliant access delegation. In embodiments involving token-based rewards or achievement tracking, user interface delivery system 112 may include components for managing user balances, triggering unlock events, and facilitating exchanges based on predefined scoring thresholds or engagement milestones.

To accommodate accessibility and multi-device usage, user interface delivery system 112 may implement responsive design principles, support screen readers, and expose simplified views for smartwatch or wearable device interfaces. In certain implementations, the system may include offline viewing capabilities or lightweight synchronization protocols to maintain functionality in low-connectivity environments. Interaction data captured from rendered interfaces may be transmitted back to competition management system 108 for further analysis, including engagement modeling, feature refinement, or interface optimization.

Model training system 114 is operable to generate, evaluate, and store machine learning models that enable handicap scoring and cohort-based normalization within a health competition platform. More specifically, model training system 114 receives structured training datasets comprising normalized health metric vectors, cohort assignment labels, demographic metadata, and device profile parameters. The system executes supervised or semi-supervised training routines to produce scoring models that learn mappings between input features and population-adjusted handicap scores. Model training system 114 is further operable to evaluate trained models using holdout datasets and performance criteria including accuracy, fairness indices, cohort consistency, and calibration error. Evaluation logic may include percentile deviation checks, bias audits across demographic segments, or comparison against historical baselines.

For example, model training system 114 may ingest a training corpus derived from rolling baselines of users aged 40-50 using smartwatch-based data sources, apply a gradient boosting framework to learn per-metric scoring adjustments, and output a serialized model object for deployment by model-based inference system 110. Model versions and performance logs may be stored in a versioned datastore for traceability and rollback. Training workflows may be scheduled periodically or triggered dynamically based on drift metrics received from downstream systems, such as competition management system 108 or another appropriate system or component.

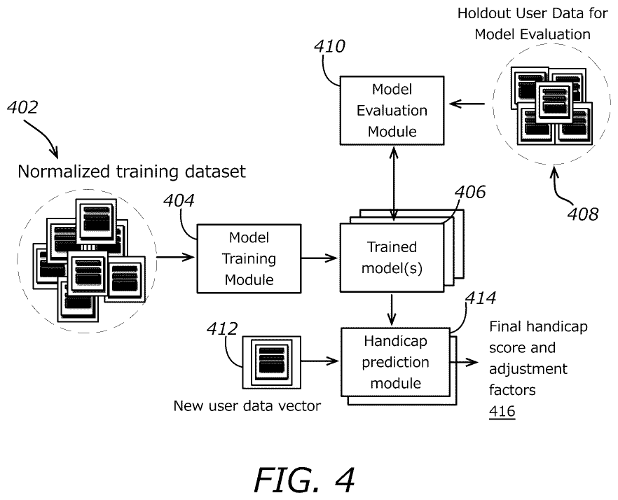

In certain embodiments, model training system 114 supports federated learning protocols, enabling model refinement using decentralized user data without centralized raw data access. Training routines may incorporate differential privacy techniques, secure aggregation logic, or device-local pretraining to preserve user privacy while improving model generalization. The training pipeline may operate in batch or streaming mode, depending on available telemetry and competition cadence. Model training system 114 will be further described in FIG. 4.

Network 120 generally represents a network or collection of networks (such as the Internet, a corporate intranet, or a combination of both) over which the various components illustrated in FIG. 1 communicate and interact, including user device(s) 102, health data acquisition system 104, external system integration gateway 106, competition management system 108, model-based inference system 110, and user interface delivery system 112. In particular embodiments, network 120 is an intranet, an extranet, a virtual private network (VPN), a local area network (LAN), a wireless LAN (WLAN), a wide area network (WAN), a metropolitan area network (MAN), a portion of the Internet, or another network 120 or a combination of two or more such networks 120. One or more links connect the systems and services described herein to network 120. In particular embodiments, one or more links each include one or more wired, wireless, or optical links.

Network 120 connects the various computing devices and systems referenced herein and facilitates the transfer of health metric data, normalized scores, model parameters, competition configurations, user preferences, and interface payloads. In various embodiments, communication between components may occur over secure channels using authenticated protocols, allowing the system to maintain data integrity and operational consistency across distributed and multi-user environments.

One or more links couple systems, services, or user devices to network 120. These links may include cloud-based APIs, secure HTTPS endpoints, persistent socket connections, or other interface protocols appropriate for structured health data transmission and competitive event coordination. In particular embodiments, the network links include an intranet, an extranet, a VPN, a LAN, a WLAN, a WAN, a MAN, a portion of the Internet, or any suitable combination of such links.

In particular embodiments, each system or engine may be implemented on a unitary server or may be distributed across multiple physical machines or containerized services. The systems described herein—including competition management system 108 and model-based inference system 110—may comprise application-layer servers, scoring engines, interface renderers, or model orchestration services. These may operate within a cloud-based architecture or a private-hosted environment and may be deployed using hardware, software, or hybrid infrastructures.

In some implementations, one or more datastores are communicatively linked to the systems described above via network 120. These datastores may persist time-series health metric records, user cohort assignments, model outputs, competition event logs, and user interaction telemetry. Storage may be implemented using relational databases, time-series databases, document stores, or hybrid architectures. Particular embodiments may expose interfaces allowing competition management system 108 and model-based inference system 110 to access, query, and update the stored data for use in cohort modeling, score generation, and user-facing visualizations.

The system may also include additional subsystems and datastores not illustrated in FIG. 1 but that would be readily understood by a person of ordinary skill in the art. For example, the system may include datastores for persisting raw and normalized health metric data, user demographic profiles, device calibration parameters, cohort assignments, competition event logs, and trained model artifacts. Other subsystems and storage components may be included or omitted depending on the deployment context or application requirements, without departing from the scope of the invention.

Accordingly, in accordance with various embodiments, the described computing system provides a technical solution to the problem of generating equitable health metric scores across heterogeneous user devices, data sources, and measurement modalities. By normalizing multi-source health data, applying device-specific calibration parameters, segmenting users into adaptive cohorts, and computing handicap-adjusted scores through machine-executed modeling logic, the system transforms unstructured and inconsistent time-series health data into standardized, cohort-referenced scoring outputs. For example, raw metric values from wearable sensors, smartphones, and third-party health platforms are processed into schema-aligned representations, baseline-adjusted, and evaluated against population-derived models to produce scores that reflect relative user performance. This transformation process addresses technical challenges related to cross-platform data inconsistency, measurement bias, and demographic variability by enabling automated, real-time generation of normalized scores that cannot be computed through manual comparison or static rule sets. The system improves the functioning of the computer by facilitating scalable, privacy-compliant, and dynamically adjustable scoring in competitive health environments.

### Competition Management System

FIG. 2 illustrates an example configuration of the competition management system 108, in accordance with various embodiments. As shown, the competition management system 108 includes health data acquisition system interface 202, external system integration gateway interface 204, model-based inference system interface 206, model training system interface 207, raw data storage system 208, normalized data storage system 210, training data repository 212, data ingestion and normalization module 214, device calibration module 216, cohort assignment module 218, baseline computation module 220, handicap adjustment module 222, score computation module 224, scoring adjustment module 226, challenge orchestration module 228, and user interface delivery system interface 230.

Health data acquisition system interface 202 is operable to facilitate structured communication between competition management system 108 and health data acquisition system 104. More specifically, health data acquisition system interface 202 manages the receipt of unstructured or semi-structured time-series health metric data from a plurality of user devices and health tracking platforms. Health data acquisition system interface 202 supports configurable transport mechanisms including RESTful APIs, streaming endpoints, and secure file upload protocols, and may enforce schema validation logic prior to internal processing. Health data acquisition system interface 202 is configured to accept varying data payload formats, including JSON, CSV, and binary telemetry blobs, and to manage ingestion timing, deduplication, and signature verification for received records.

For example, health data acquisition system interface 202 may receive timestamped step count vectors from a wearable pedometer, continuous heart rate telemetry from a chest strap monitor, and sleep cycle summaries from a third-party mobile application, each in a distinct payload structure. Health data acquisition system interface 202 identifies the source device type, user association, and health metric category, tags each record with a system-level identifier, and transmits the payload to data ingestion and normalization module 214 for schema alignment. In certain embodiments, health data acquisition system interface 202 supports asynchronous ingestion via queue-based message brokers, enforces per-user ingestion quotas, and performs checksum comparisons for record-level integrity enforcement.

In some implementations, health data acquisition system interface 202 includes metadata tagging logic to assign device identifiers, ingestion timestamps, and origin flags to each received payload, enabling traceability and reproducibility throughout subsequent processing stages. Health data acquisition system interface 202 may operate in coordination with access control layers to validate user consent tokens, device authorization keys, or federation credentials prior to accepting metric data from remote endpoints. Health data acquisition system interface 202 is operable across heterogeneous device ecosystems and may normalize communication differences between consumer-grade health trackers, fitness equipment sensors, and clinical monitoring devices through device-specific protocol handlers.

In certain embodiments, health data acquisition system interface 202 is further operable to extract or assign activity modality attributes based on device type, source application, embedded context metadata, or signal characteristics. For example, metric records may be annotated to distinguish between treadmill running, outdoor running, stationary cycling, or elliptical use, using device identifiers or environmental context indicators. These modality indicators are incorporated into structured metadata fields and preserved throughout normalization and baseline modeling operations. The resulting tagged records support intra-modality comparisons (e.g., consistent activity type), modality-variant comparisons (e.g., treadmill versus outdoor running), and inter-modality comparisons (e.g., running versus cycling), enabling the downstream scoring architecture to adjust for modality differences across scoring and handicap layers.

External system integration gateway interface 204 is operable to facilitate data exchange between competition management system 108 and external system integration gateway 106. More specifically, external system integration gateway interface 204 enables secure, structured communication with third-party platforms, services, and cloud-based data providers to augment health metric records with supplemental context or to retrieve federated user information. External system integration gateway interface 204 supports authenticated communication protocols such as OAuth 2.0, SAML, or API key validation, and may expose platform-specific integration adapters for interfacing with external fitness aggregators, medical record providers, employer wellness systems, or data marketplaces. The received third-party attributes may include age, gender, device certification parameters, activity modality context, or known medical flags—each of which may contribute to downstream demographic-based scoring adjustment operations.

For example, external system integration gateway interface 204 may retrieve user demographic metadata from a federated identity provider, fetch historical training data from a third-party fitness cloud, or pull environmental condition logs (e.g., air quality, weather, geolocation) from a location-aware data service. Each received payload may be validated for structure, timestamp alignment, and user association before routing to downstream components within competition management system 108. In certain embodiments, external system integration gateway interface 204 supports scheduled polling and event-driven push updates, enabling time-coordinated ingestion of third-party metrics or adjustments from asynchronous sources. The retrieved data may be tagged with modality indicators or cohort-alignment parameters and transmitted to cohort assignment module 218 and handicap adjustment module 222 to support demographic and modality-aware normalization.

In some implementations, external system integration gateway interface 204 includes routing logic to map incoming records to one or more internal subsystems, including device calibration module 216 or cohort assignment module 218. External system integration gateway interface 204 may maintain a registry of active external connections, including data source identifiers, access credentials, protocol endpoints, schema definitions, and rate limits. In certain embodiments, external system integration gateway interface 204 includes compliance auditing logic to log all data exchanges, record user consent associations, and enforce region-specific data residency rules in accordance with applicable privacy regulations. These compliance logs may be used to validate eligibility for cohort scoring, demographic normalization, or device-specific calibration routines.

Model-based inference system interface 206 is operable to manage data exchange between competition management system 108 and model-based inference system 110. More specifically, model-based inference system interface 206 transmits schema-aligned, calibrated metric vectors and associated user metadata for predictive modeling, cohort classification, and handicap adjustment inference. The returned outputs may include dynamically inferred population cohort assignments, per-metric adjustment vectors aligned with user-specific deviations, cohort-normalized reference parameters, and drift indicators relevant to retraining logic. These outputs are consumed by downstream modules of competition management system 108 to support generation of handicap-adjusted scores based on personal baselines, demographic alignment, and composite scoring weights.

In various embodiments, model-based inference system interface 206 transmits structured feature vectors derived from rolling user baselines (Handicap 1), device calibration parameters and demographic coefficients (Handicap 3), and current activity metrics to model-based inference system 110. These feature vectors serve as inputs to the cohort modeling engine and handicap modeling engine described in FIG. 3, which generate inferred scoring adjustments, similarity-based cohort assignments, and configuration-specific handicap factors. Model-based inference system interface 206 supports both batch inference requests and low-latency asynchronous queries, and may implement message-oriented middleware using gRPC, Apache Kafka, or RESTful JSON protocols over secure HTTPS sessions.

Model-based inference system interface 206 may also transmit retraining triggers, cohort drift metrics, or scoring variance indicators to model-based inference system 110 based on metrics received from components such as cohort assignment module 218 or score computation module 224. For example, instability in cohort distribution or sustained deviation bias may initiate partial retraining of the handicap modeling logic to realign scoring fairness across population segments. In certain implementations, model-based inference system interface 206 appends authentication headers, model version identifiers, and user consent state indicators to each transmission. The interface may log inbound feature vectors, returned model predictions, and drift events to support auditability, model version rollback, and population-level performance tracking.

Model training system interface 207 is operable to facilitate machine-executed data transmission between competition management system 108 and model training system 114. More specifically, model training system interface 207 enables the export of training datasets comprising normalized health metric vectors, user demographic profiles, assigned cohort labels, and calibration metadata generated by internal components of competition management system 108. These training datasets are used to develop and refine predictive scoring models supporting the handicap adjustment logic shown in FIG. 3. The interface supports bidirectional communication to receive model evaluation results, performance metrics, and retraining triggers initiated by cohort drift or scoring instability indicators.

For example, model training system interface 207 may transmit structured feature vectors extracted from training data repository 212 to model training system 114. These feature vectors may encode rolling user baselines used for Handicap 1, demographic and device calibration indicators relevant to Handicap 3, and aggregated metric-level scoring outcomes aligned to Handicap 2 weighting logic. The interface may also receive serialized model artifacts—such as trained cohort classifiers or per-metric adjustment models—along with associated validation statistics (e.g., deviation error, fairness index, demographic parity score). The received model objects may include version identifiers and are registered with scoring modules of competition management system 108 for operational deployment.

In certain embodiments, model training system interface 207 includes job queuing logic for asynchronous retraining execution, access control gates for training data write privileges, and telemetry feedback loops to ensure successful transmission and ingestion of structured training records. The interface may also publish training job completion signals, which are used to trigger version updates or scoring recalibration workflows across cohort assignment module 218, handicap adjustment module 222, and score computation module 224. These mechanisms support automated model lifecycle management, ensuring that handicap modeling remains responsive to evolving user behavior, population dynamics, and metric drift.

Raw data storage system 208 is operable to persist unstructured and semi-structured health metric data ingested from user devices, third-party platforms, and contextual data providers prior to normalization, calibration, or cohort processing. More specifically, raw data storage system 208 stores heterogeneous health metric inputs received via health data acquisition system interface 202 and external system integration gateway interface 204. These inputs may include time-series metric values (e.g., heart rate, step count, exertion level, or sleep stages), device metadata, ingestion timestamps, and transmission context originating from wearable devices, fitness equipment, or software-based activity trackers.

For example, raw data storage system 208 may persist one-minute interval accelerometer data received from a smartwatch, sub-second heart rate telemetry streamed from a chest strap monitor, and user-annotated sleep cycle summaries uploaded from a mobile health application. Each record may be stored in its native or semi-structured payload format (e.g., JSON, XML, protobuf), accompanied by device identifiers, ingestion flags, and timestamped transmission metadata. In certain embodiments, raw data storage system 208 organizes records by user ID, device class, and ingestion window using partitioned key structures to enable efficient downstream access by normalization and calibration modules.

In some embodiments, raw data storage system 208 includes ingestion trace logging and data integrity enforcement. For example, the system may apply hash-based verification during storage to confirm record completeness, maintain append-only ingestion logs for traceability, and store source-tagged metadata to support reproducibility and data lineage tracking. Raw data storage system 208 interfaces with data ingestion and normalization module 214 and device calibration module 216 to retrieve payload subsets for schema alignment, metric standardization, and calibration transformations, while preserving the original ingested records for auditability, reprocessing, or future model retraining workflows.

In various embodiments, the unstructured health metric data ingested and stored by raw data storage system 208 includes physiological and behavioral biomarkers used for individualized scoring. Such biomarkers may include time-series records of cardiovascular indicators (e.g., heart rate, heart rate variability), respiratory signals (e.g., blood oxygen saturation), sleep architecture features (e.g., REM duration, deep sleep percentage), metabolic values (e.g., glucose levels or caloric expenditure), and physical performance metrics (e.g., VO2 max estimates, gait cadence, or peak exertion). These biomarkers may be derived from biosensor-equipped devices (e.g., chest strap monitors, optical HR trackers, or sleep bands), clinical integrations (e.g., blood test results received via secure health APIs), or user-reported digital assessments. Each ingested biomarker may be associated with metadata including measurement intervals, sensor fidelity, and ingestion context (e.g., device type, user activity state, or environmental conditions), enabling precise downstream calibration and interpretation. The system utilizes these biomarkers as foundational inputs to compute user-specific baselines, evaluate cohort deviation vectors, and generate normalized handicap scores reflective of observed physiological function.

Normalized data storage system 210 is operable to store schema-conformant representations of health metric data after unit harmonization, field mapping, and temporal alignment operations have been performed by data ingestion and normalization module 214. More specifically, normalized data storage system 210 receives structured metric records that include standardized values, canonical field names, device-agnostic timestamps, and source-traceable metadata aligned to an internal schema supporting multi-device health scoring workflows.

For example, normalized data storage system 210 may store per-user metric vectors aligned to five-minute sampling intervals, where each record includes converted values for metrics such as step rate, heart rate, sleep duration, or energy expenditure. These values may be expressed in harmonized units (e.g., steps per minute, beats per minute, kilocalories), mapped to internal schema fields (e.g., step_rate_mpm, resting_hr_bpm), and tagged with transformation metadata including fidelity scores, source tags, and transformation hashes enabling traceability to raw inputs stored in raw data storage system 208.

In certain embodiments, normalized data storage system 210 implements vectorized storage formats and time-series indexing structures optimized for downstream access by cohort assignment module 218, baseline computation module 220, handicap adjustment module 222, and model-based inference system 110. Storage backends may include columnar data warehouses (e.g., Apache Parquet, Delta Lake) or time-series databases (e.g., InfluxDB, TimescaleDB) configured for efficient retrieval of metric sequences across rolling time windows or cohort-based groupings. In distributed implementations, normalized data storage system 210 may replicate schema-aligned datasets to edge compute environments or federated storage nodes via external system integration gateway interface 204.

Normalized data storage system 210 enables machine-executed operations to resolve semantic, structural, and temporal inconsistencies across heterogeneous health metric sources. These operations address the technical problem of data fragmentation across device ecosystems by transforming unstructured health metric data into schema-conformant datasets usable for algorithmic calibration, cohort-based modeling, and scalable scoring pipelines.

Training data repository 212 is operable to store curated, schema-conformant health metric data and associated user metadata for use in training, validating, and updating machine learning models within model-based inference system 110 and model training system 114. More specifically, training data repository 212 stores structured feature vectors, rolling baseline trends, cohort assignment labels, and calibration parameters derived from upstream processing stages in competition management system 108.

In various embodiments, training data repository 212 stores per-user feature vectors that include calibrated metric sequences (e.g., smoothed heart rate trends, time-normalized step vectors), contextual demographic attributes (e.g., age, device model, prior cohort ID), and label values derived from expert annotations, outcome-based scoring, or model-generated classifications. These labeled vectors may support training of supervised or semi-supervised models used for cohort segmentation, handicap adjustment scoring, or drift detection. Repository records may be partitioned by time window, demographic strata, or device ecosystem to support targeted retraining and fairness analysis.

Training data repository 212 may also store holdout partitions for validation and generalization testing. For example, the repository may reserve user datasets excluded from training pipelines for use in cross-validation procedures, A/B testing of candidate models, or scoring fairness evaluation. In certain implementations, training data repository 212 maintains versioned snapshots of training datasets, enabling reproducible training workflows and rollback in the event of drift or model degradation.

Training data repository 212 supports machine-based model training workflows by transforming incoming user metric streams into structured, retrainable datasets. These machine-executed operations enable adaptive learning pipelines, reproducible cohort modeling, and context-aware scoring adjustments across diverse health tracking sources. The repository addresses the technical problem of adapting scoring models to dynamic user populations by enabling historical traceability, scalable retraining logic, and structured data partitioning for fairness-aligned optimization.

Data ingestion and normalization module 214 is operable to receive heterogeneous health metric data from health data acquisition system interface 202, external system integration gateway interface 204, and model training system interface 207, and to transform the data into schema-conformant metric records suitable for calibrated scoring and cohort modeling. More specifically, data ingestion and normalization module 214 performs unit normalization, temporal alignment, field resolution, and schema enforcement to generate standardized metric vectors from raw or semi-structured input payloads.

In various embodiments, data ingestion and normalization module 214 includes a parsing engine configured to extract time-series values, metadata fields, and contextual indicators based on device-specific signatures or payload headers. For example, the module may map inconsistent step count fields labeled “steps,” “stride_total,” or “sc” to a canonical field; similarly, it may reconcile sleep metrics recorded as “asleep_time,” “total_sleep,” or “sleep_minutes” into a unified format. The parsing logic applies deterministic mappings, device-specific rules, or learned transformation tables to resolve data variability across input schemas.

Data ingestion and normalization module 214 performs unit conversion and sampling normalization across disparate devices and metric types. For example, heart rate may be reported in beats per minute or in millisecond intervals between beats; the module converts all representations to bpm. For sleep staging, data reported using N1/N2/N3/REM/wake or light/deep/REM schemas may be mapped into a unified four-state representation with aligned sampling intervals. Sampling normalization techniques include fixed-window resampling, weighted averaging, or linear interpolation based on resolution consistency rules.

In certain embodiments, data ingestion and normalization module 214 enforces a configurable schema model that defines required metric fields, allowed value ranges, and structural constraints. Schema enforcement may validate timestamp order, check metric consistency (e.g., total sleep time ≤total recording time), and ensure physiological plausibility. Invalid records may be flagged, discarded, or logged for downstream inspection. The module also applies tagging logic to preserve lineage metadata for each transformed record, enabling traceability through calibration, scoring, and modeling stages.

Data ingestion and normalization module 214 enables machine-based transformation of semantically inconsistent and device-divergent input data into interoperable metric vectors suitable for calibration and cohort scoring. These operations address the technical challenge of harmonizing variably formatted time-series health records across diverse ecosystems, and they form the basis for structured, reproducible scoring across individualized and population-based handicap models.

Device calibration module 216 is operable to apply device-specific correction parameters to normalized health metric vectors generated by data ingestion and normalization module 214. More specifically, device calibration module 216 transforms each metric value using a calibration profile associated with the originating tracking device, where the profile includes correction coefficients, fidelity indicators, sensor latency offsets, and error distribution models derived from empirical validation or device manufacturer data.

In various embodiments, device calibration module 216 accesses a structured repository of device profiles, each containing parameterized correction values tied to known measurement biases. For example, a smartwatch known to underreport heart rate at high exertion levels may be assigned a +3.2 bpm offset during segments exceeding a specified activity threshold. A sleep tracker with coarse granularity may include a −5-minute correction for sleep onset. Device calibration module 216 applies these corrections using per-metric adjustment logic, optionally incorporating cross-variable effects such as device type and demographic interaction (e.g., optical sensor sensitivity vs. skin tone).

Device calibration module 216 may further support adaptive calibration based on aggregated user telemetry or time-aligned feedback signals. For instance, when multiple users exhibit consistent undercounting during treadmill incline sessions on a specific device model, the module may update the correction logic to apply an activity-conditional multiplier for future data segments. Calibration logic may be encoded as fixed-function rules, lookup-based mappings, or learned regression layers trained on labeled datasets collected under known reference conditions.

In certain embodiments, device calibration module 216 generates confidence scores that reflect the quality or reliability of each corrected metric value. These scores may account for data completeness, correction factor stability, or underlying sensor fidelity and may be embedded as metadata fields within the calibrated vector. Downstream modules—such as baseline computation module 220 and handicap adjustment module 222—may use these confidence indicators to inform scoring weight or adjustment sensitivity. Device calibration module 216 thereby addresses the technical problem of inter-device measurement inconsistency by transforming device-normalized values into calibrated representations suitable for equitable scoring.

Cohort assignment module 218 is operable to determine population-based cohort groupings for each user based on calibrated metric vectors, demographic attributes, biometric indicators, and device metadata. More specifically, cohort assignment module 218 is configured to execute schema-conformant classification logic that assigns each user to a dynamic cohort used to inform deviation modeling, baseline normalization, and handicap score generation. The cohort assignment process enables structured mapping of users to comparable peer groups across one or more similarity dimensions, including physiological, behavioral, environmental, and contextual factors.

In various embodiments, cohort assignment module 218 receives calibrated metric records and associated user metadata from device calibration module 216 and normalized data storage system 210. The user metadata may include demographic values (e.g., age, sex, height), biometric attributes (e.g., resting heart rate, blood type, skin tone, or sleep chronotype), and contextual indicators (e.g., altitude, weather conditions, circadian rhythm alignment). Cohort assignment module 218 applies deterministic, rule-based, or model-inferred classification logic to compute one or more cohort assignment labels per user. These labels serve as references for comparative analysis and scoring normalization during handicap adjustment workflows.

For example, cohort assignment module 218 may classify a 48-year-old male user with a known sleep disorder and smartwatch-derived heart rate metrics into a baseline cohort defined by males aged 45-55, using optical sensors, with low REM sleep efficiency. This assignment supports personalized deviation modeling in the case of intra-activity comparisons (e.g., comparing today's outdoor cycling to the user's own past outdoor cycling sessions), peer-normalized adjustments across users performing similar activities, or demographically normalized scoring for fairness across device and physiological baselines. Cohorts may be defined by discrete clusters (e.g., k-means or quantile bands) or continuous mappings (e.g., z-score thresholds or similarity distances) depending on system configuration.

In some implementations, cohort assignment module 218 supports multi-dimensional assignment logic for resolving comparative scenarios across different activity modalities. For instance, where a user performs treadmill running on one day and outdoor running on another (a variation of the same activity modality), cohort assignment module 218 may apply similarity modeling to associate the current vector with a derived hybrid cohort representing treadmill-adjusted outdoor metrics. In other implementations, if the user switches from running to rowing (a structurally distinct activity class), cohort assignment module 218 may assign the user to a distinct modality cohort while retaining biometric and demographic alignment.

In certain embodiments, cohort assignment module 218 includes real-time cohort reassignment triggers that respond to changes in baseline performance, updated health conditions, or periodic device upgrades. The module may recompute cohort labels based on new device metadata, such as a switch from wrist-based sensors to chest straps, or on observed physiological transitions, such as sleep chronotype shifts or hormonal phase changes. These triggers ensure that comparative scoring operations remain aligned with evolving user characteristics and measurement conditions.

Cohort assignment module 218 enables machine-executed groupings of users into schema-normalized peer clusters used for modeling deviation scores, applying handicap adjustments, and generating equitable competition scores across heterogeneous users and devices. These assignments support structured processing pipelines for comparative baseline generation, population-aware scoring calibration, and dynamic handicap adjustment across personal improvement, relative competition, and demographic normalization dimensions.

Baseline computation module 220 is operable to generate rolling baseline vectors for individual users based on normalized, calibrated health metric records retrieved from normalized data storage system 210. More specifically, baseline computation module 220 processes historical time-series values for a plurality of health-related metric types—such as heart rate variability, step count, sleep duration, respiratory rate, and energy expenditure—to construct multi-dimensional baseline vectors that represent a user's recent physiological state over a configurable time window (e.g., 7-day, 14-day, or 28-day interval). These baselines serve as individualized reference models for comparison against current metric inputs, cohort benchmarks, and adjusted scoring parameters.

In various embodiments, baseline computation module 220 segments incoming time-series vectors by activity modality, sleep stage classification, or user-defined goal category to produce modality-specific baselines for distinct activity types. For example, baseline computation module 220 may generate separate rolling baselines for treadmill walking versus outdoor hiking, or for REM sleep duration versus total sleep time, depending on the granularity of the input signal and associated metadata tags. These baselines may be aligned with modality identifiers extracted during normalization or inferred via activity classification logic embedded in earlier processing stages.

Baseline computation module 220 applies configurable statistical operators to each metric vector, including moving averages, exponential smoothing, interquartile thresholds, and anomaly masking to reduce the influence of outliers, transient spikes, or invalid readings. For example, in constructing a step count baseline, the module may discard values from days when total recorded duration falls below a minimum threshold (e.g., 8 hours of device uptime), apply a weighted average to favor more recent days, and interpolate missing values using local trend estimates. These techniques ensure that the resulting baseline is both temporally representative and robust to variation across days or device conditions.

In certain embodiments, baseline computation module 220 incorporates device calibration metadata and user demographic attributes into the baseline generation logic. For instance, the module may apply age-specific decay coefficients when smoothing heart rate variability baselines or adjust sleep quality baselines based on known device resolution for sleep staging intervals. This integration enables the system to generate normalized baseline vectors that are resilient to measurement biases and physiological diversity across users, supporting consistent scoring across heterogeneous input sources.

Baseline computation module 220 is further operable to generate output representations in the form of normalized deviation maps, which quantify the difference between a user's current metric vector and their corresponding baseline vector. These deviation maps are passed to downstream scoring modules—including the handicap adjustment module 222—for use in generating individualized improvement scores, adjusted performance values, and cohort-relative comparisons. In embodiments involving competition formats with cross-activity comparisons, baseline computation module 220 generates multiple baseline tracks per user, allowing for weighted or aligned comparisons across activity variants or category transitions (e.g., cycling-to-rowing, walking-to-stair climbing).

The machine-executed transformations performed by baseline computation module 220 —namely, converting raw metric sequences into baseline vectors with embedded normalization, smoothing, modality disambiguation, and demographic correction—enable downstream modules to compute performance improvements without reliance on static thresholds or external benchmarks. These transformations are performed using structured time-series pipelines operating over schema-conformant input data, allowing for repeatable, scalable, and privacy-preserving baseline generation across large user populations.

Handicap adjustment module 222 is operable to generate handicap adjustment vectors for a user by applying individualized scoring corrections to health metric data based on prior performance history, demographic segmentation, and cohort-relative normalization. More specifically, handicap adjustment module 222 is configured to compute a set of multi-factor adjustment values for a given user and activity context, using as input (i) modality-specific baseline vectors generated by baseline computation module 220, (ii) cohort reference vectors received from cohort assignment module 218, and (iii) context attributes including demographic attributes, activity type, and device profile metadata.

In various embodiments, handicap adjustment module 222 applies a multi-layer scoring transformation that includes baseline-relative adjustments, cross-user normalization, and demographically weighted corrections. Each layer of transformation operates on structured, schema-conformant metric vectors, and applies parameterized logic to generate a per-metric adjustment component. For example, the module may compute a delta between the user's current heart rate variability and their individualized rolling baseline, normalize the result against cohort reference statistics for the same activity modality, and apply a demographic scaling factor based on age or chronotype to yield an adjustment value. Similar computations may be performed for other metrics such as step cadence, sleep efficiency, or exertion load.

Handicap adjustment module 222 supports both within-modality and cross-modality comparisons. For within-modality scoring, the module applies adjustments using cohort references specific to the user's current activity (e.g., comparing treadmill running performance against treadmill cohort baselines). For cross-modality normalization, the module accesses transformation profiles that map performance metrics across similar or alternate activity types (e.g., mapping elliptical training metrics to equivalent treadmill cadence zones), enabling consistent scoring logic even when users perform different but related activities during a scoring interval.

The output of handicap adjustment module 222 is a structured adjustment vector comprising one or more weighted scoring corrections associated with the user's current metrics. Each correction includes metadata tags such as source modality, cohort label, confidence score, and calibration trace. The adjustment vector is passed to score computation module 224 for application to the raw or calibrated input metrics, resulting in a normalized score used for competition ranking.

In certain embodiments, handicap adjustment module 222 supports configurable adjustment pipelines that define the order and interaction of adjustment layers. For example, the system may first apply a baseline-relative improvement layer, followed by a cohort alignment layer, and finally a demographic normalization layer. Each stage may incorporate rule-based or model-based logic and may be tuned via parameters stored in scoring configuration profiles. These profiles may be selected based on competition format, activity type, or organizational scoring policy.

In some implementations, handicap adjustment module 222 includes safeguards for adjustment bias mitigation. For example, the system may evaluate fairness indices across demographic subgroups and apply constraint logic to ensure adjustment distributions remain within acceptable equity bounds. Additionally, the module may encode retraining signals based on drift detection—such as when adjustment distributions exhibit persistent asymmetry across population segments—and forward these signals to model-based inference system 110 or model training system 114.

The adjustment operations performed by handicap adjustment module 222 enable the system to transform heterogeneous, device-dependent health metric values into calibrated, fair, and competition-compliant scoring inputs. These structured, machine-executed transformations address technical challenges associated with scoring consistency, user variability, and device heterogeneity, and provide claim-aligned support for generating personalized handicap vectors using user baseline data, cohort references, and demographic attributes.

Score computation module 224 is operable to generate a normalized score for a user based on the application of one or more handicap adjustment vectors to calibrated health metric data. More specifically, score computation module 224 receives a user's current metric vector, one or more handicap adjustment vectors generated by handicap adjustment module 222, and scoring configuration parameters, and applies these inputs to compute a scalar score value that reflects the user's relative performance within a defined scoring context.

In various embodiments, score computation module 224 applies an aggregation function to combine multiple per-metric adjustment components into a composite score. The aggregation function may be configured to perform weighted summation, multiplicative blending, or nonlinear transformation of the individual components, with weights defined according to user preferences, competition format, metric reliability, or scoring policy. For example, the system may assign higher weights to exertion metrics (e.g., average heart rate elevation) during endurance challenges, or to recovery metrics (e.g., sleep efficiency, heart rate variability) during rest-based competitions.

Each adjustment component may correspond to a specific scoring dimension—for instance, deviation from a modality-specific baseline, distance from a cohort reference range, or alignment with expected demographic norms. Score computation module 224 computes the per-metric delta, applies correction weights, and produces an intermediate scoring contribution. These intermediate contributions are aggregated into a final normalized score for the user and may include sub-scores or annotations reflecting which adjustment layers were applied.

For activity comparisons within the same modality, score computation module 224 aligns baseline and adjustment inputs based on temporal resolution and event-type tagging (e.g., 5-minute blocks for walking sessions). For comparisons across related modalities, the module applies modality bridging logic to translate metric types into a shared vector space. For example, the system may normalize cadence metrics from treadmill runs and elliptical sessions using activity-normalized pacing coefficients, allowing users who performed different exercises to be scored on a unified scale.

In certain embodiments, score computation module 224 supports dynamic scoring pipelines with tunable aggregation parameters. These parameters may include adjustment caps, per-metric floors, transformation thresholds, and weighting policies configured per competition. For example, in a population-wide competition spanning multiple time zones and device ecosystems, the module may downweight low-confidence metrics (e.g., poor-quality sleep staging data from uncalibrated devices) while emphasizing consistently captured metrics like step count or time-in-zone heart rate.

Score computation module 224 also includes versioning logic for scoring profiles, allowing the system to track and apply distinct scoring rulesets across different challenges or time periods. This supports longitudinal comparisons and historical progress tracking for users while preserving consistency within a given competition window.

In some implementations, score computation module 224 generates auxiliary scoring metadata, including confidence intervals, normalization traces, and cohort-relative percentiles, which are embedded in the score output structure and passed to user interface delivery system 112 for presentation. Additionally, the module logs scoring events, model version tags, and input hashes to a scoring event repository, enabling full traceability and reproducibility of the scoring output.

Score computation module 224 enables machine-executed transformation of individualized metric data and adjustment vectors into structured competition scores suitable for leaderboard ranking, progress feedback, and reward distribution. The structured composition and aggregation logic of the module ensures that scoring is based on calibrated, context-aware, and demographically aligned inputs, enabling consistent and fair comparisons across heterogeneous activities, users, and device types.

Scoring adjustment module 226 is operable to modify a computed score based on context-sensitive parameters, comparative performance scaling, and population-wide fairness criteria. More specifically, scoring adjustment module 226 receives a normalized score generated by score computation module 224 and applies one or more machine-executed transformation functions to adjust the score prior to presentation, ranking, or reward determination.

In various embodiments, scoring adjustment module 226 applies comparative normalization operations based on aggregate competition data, including mean and standard deviation of cohort scores, percentile thresholds, or competition-specific reference benchmarks. For example, in a team-based challenge where user scores are aggregated by subgroup, the system may adjust individual scores upward or downward based on intra-team variability or performance distribution skew, ensuring consistent contribution scaling across uneven teams.

Scoring adjustment module 226 is further configured to apply context-aware transformation policies that align with the structure of the user's activity data, demographic segment, and device ecosystem. For example, the module may apply linear scaling for users whose scoring range has been compressed due to underreporting by lower-fidelity devices, or apply sigmoid-based compression to limit score inflation in high-performing cohorts. These transformations are parameterized by stored calibration metadata and are executed using per-user transformation functions, ensuring user-specific reproducibility.

In certain implementations, scoring adjustment module 226 applies temporal reweighting factors that incorporate rolling historical performance or multi-session engagement signals. For instance, a user's current score may be adjusted based on a 7-day moving average of participation consistency or improvement velocity, introducing momentum-based modulation into the competition outcome. In one example, users who show rapid improvement relative to their own historical norm may receive adjustment boosts, while those with anomalous score spikes may be subject to dampening coefficients derived from variance thresholds.

Scoring adjustment module 226 may also implement fairness-oriented transformation routines, using demographic-specific coefficients stored in a scoring policy profile. These routines are configured to reduce structural disparities between population groups and account for known physiological, behavioral, or device-related performance constraints. For example, scoring curves may be adjusted to normalize peak heart rate targets across age brackets or to equalize motion detection sensitivity across different wearable sensor classes.

To support transparency and auditability, scoring adjustment module 226 may generate a scoring lineage record that logs the raw score, each adjustment function applied, all intermediate values, and the final adjusted score. This trace is stored in a scoring audit repository and may be made available to model-based inference system 110 to monitor drift in scoring fairness or identify adjustment patterns requiring retraining.

In some embodiments, scoring adjustment module 226 supports tunable adjustment modes that can be configured per competition type, user group, or metric category. The mode selection may be driven by challenge parameters retrieved from challenge orchestration module 228 and may dictate which transformation layers are active. For example, a head-to-head format may enable only relative scaling adjustments, while a multi-team enterprise challenge may activate group-normalized and demographic-equity transformations.

Scoring adjustment module 226 enables structured modification of user scores using parameterized transformation functions that are programmatically defined, data-driven, and traceable across population contexts. These machine-executed operations ensure that final scoring values reflect not only raw performance but also dynamic contextual alignment and population-aware calibration, supporting equitable and robust comparison across heterogeneous data environments.

Challenge orchestration module 228 is operable to define, configure, and manage competitive scoring challenges across a distributed population of users. More specifically, challenge orchestration module 228 generates structured competition records that specify eligibility constraints, scoring rules, metric weighting parameters, temporal boundaries, and performance normalization modes, and coordinates their execution across relevant subsystems within competition management system 108.

In various embodiments, challenge orchestration module 228 includes a configuration engine that receives challenge parameters from user interface delivery system 112 or an administrative backend, validates the parameters against system-supported schemas, and stores the resulting definition in a challenge configuration repository. Parameters may include start and end timestamps, included metric categories (e.g., step count, sleep duration, heart rate variability), cohort segmentation strategy, comparison mode (e.g., individual, team, group-normalized), and scoring aggregation logic (e.g., weighted mean, top-N contribution).

Challenge orchestration module 228 is further operable to apply cohort-aware configuration templates during challenge initialization. For example, when launching a challenge across multiple user cohorts, the module may automatically instantiate parallel challenge instances with cohort-specific reference models, baseline alignment factors, or scoring transformation weights. Each instance may operate under the same scoring architecture but apply different normalization profiles to ensure fair competition among demographically or behaviorally distinct populations.

In certain implementations, challenge orchestration module 228 generates metric selection policies for each challenge instance based on competition type and available user data. For instance, in an all-activity challenge, the system may allow device-level modality tags to dynamically determine the most frequently used activity category for each user and bind scoring logic to that category during the challenge duration. Alternatively, in a structured wellness program with predefined metric targets, the module may enforce static inclusion of sleep regularity and recovery index as required inputs for scoring eligibility.

Challenge orchestration module 228 may transmit challenge configuration records to cohort assignment module 218 for initial population segmentation, to baseline computation module 220 for determining the appropriate rolling window, and to score computation module 224 for loading metric-specific aggregation weights and reference comparison logic. For example, a challenge that rewards daily improvement may assign higher weighting to recent baseline deviation, while a consistency-based challenge may favor longitudinal stability in rolling averages.

To accommodate multi-user challenge formats, challenge orchestration module 228 is operable to register and track group formations, including team rosters, roles, and inter-team comparison logic. These configurations may be used by scoring adjustment module 226 to apply inter-group normalization or tie-breaking logic. In one embodiment, team contributions may be calculated using a hybrid scoring model that blends per-user improvement scores with an adjustment for intra-team variance, thereby rewarding balance and engagement consistency across the team rather than raw aggregate score.

In some embodiments, challenge orchestration module 228 includes a progression tracking subsystem that maintains real-time challenge state, such as active participant lists, current leaderboard snapshots, and completion flags. This subsystem may periodically poll or receive push updates from score computation module 224 and scoring adjustment module 226 to update challenge records and drive interface rendering through user interface delivery system 112. The state may include time-elapsed indicators, milestone triggers, and per-user reward eligibility flags.

Challenge orchestration module 228 may also be configured to enforce entry conditions for challenge eligibility, such as minimum data completeness, demographic inclusion filters, or device calibration confidence thresholds. These conditions are evaluated by coordination logic that interfaces with device calibration module 216, training data repository 212, and user demographic attributes retrieved via external system integration gateway interface 204. In some implementations, users failing to meet eligibility criteria are deferred into shadow cohorts or provided alternative challenges with relaxed scoring constraints.

Challenge orchestration module 228 enables structured, machine-defined competition environments that align individual metric data with dynamic scoring logic and context-aware normalization workflows. The orchestration of challenge parameters across scoring, cohorting, and adjustment modules ensures consistent operation of the platform's scoring architecture while allowing flexible definition of competition formats, eligibility gates, and fairness-enhancing configurations.

User interface delivery system interface 230 is operable to coordinate structured communication between competition management system 108 and user interface delivery system 112 to enable the presentation of normalized scores, cohort-adjusted outputs, and individualized user performance data on client-facing devices. More specifically, user interface delivery system interface 230 transmits formatted scoring records, adjustment vectors, cohort assignment metadata, and challenge progress indicators from internal scoring components to remote display subsystems configured to render visualizations on user device(s) 102.

In various embodiments, user interface delivery system interface 230 includes a payload formatting engine operable to transform internally stored or computed data—such as rolling metric baselines, deviation scores, and handicap-adjusted composite scores—into interface-ready data packets conforming to the rendering specifications of web or mobile applications. These data packets may include field-tagged score vectors, normalization factors, calibration indicators, and leaderboard metadata, each formatted in JSON or protocol buffer structures suitable for efficient delivery and parsing on resource-constrained clients.

User interface delivery system interface 230 may operate in push or pull configurations, supporting both client-initiated polling and server-initiated update streams. For example, during an ongoing multi-day challenge, user interface delivery system interface 230 may trigger periodic score refreshes aligned with completion of new scoring cycles, transmitting updated percentile ranks, score trajectories, or adjustment factor breakdowns to ensure interface consistency. Interface data payloads may include embedded identifiers specifying scoring mode, cohort assignment context, or calibration confidence, allowing client applications to selectively render content based on user eligibility or display configuration.

In certain implementations, user interface delivery system interface 230 transmits disaggregated performance metrics to allow client applications to render modular visualizations for improvement tracking, cohort-relative positioning, or metric-specific performance over time. For instance, a user may receive a breakdown of their per-metric improvement scores, their aggregate normalized rank within a cohort, and their deviation from predicted calibration-adjusted targets. These elements are produced by score computation module 224, scoring adjustment module 226, and baseline computation module 220 and passed through interface 230 in a structured, schema-aligned format.

User interface delivery system interface 230 may also receive user-generated input transmitted through rendered interfaces and route the input to appropriate components within competition management system 108. This input may include challenge enrollment selections, device linking actions, demographic updates, or health goal declarations. In one example, a user may manually select a preferred scoring metric bundle (e.g., sleep +step count), which is received via user interface delivery system 112 and routed through interface 230 to challenge orchestration module 228 for inclusion in a challenge instance.

To maintain privacy compliance and user-specific access controls, user interface delivery system interface 230 may append identity tokens, session metadata, and consent tags to all transmitted payloads. These tags are validated prior to transmission and are preserved across the interface boundary to ensure that personalized scoring information, cohort classification status, and handicap-adjusted outputs are only accessible to authorized users or their delegated agents. In some embodiments, user interface delivery system interface 230 integrates with federated identity services via external system integration gateway interface 204 to support single sign-on, role-based rendering, and privacy-compliant data access delegation.

User interface delivery system interface 230 is further operable to transmit interface configuration directives that tailor rendering logic based on scoring mode, challenge type, or device capabilities. For example, the system may signal that percentile-based cohort visualization should be used for comparative challenges, while progress bar trackers should be enabled for consistency-focused scoring formats. These rendering hints are generated based on metadata produced by challenge orchestration module 228 and scoring adjustment module 226, ensuring consistency between back-end scoring logic and front-end display semantics.

Through its structured bidirectional exchange capabilities, user interface delivery system interface 230 enables machine-generated scoring outputs to be presented in a format interpretable by end users and allows user-initiated configuration or feedback to be reflected in ongoing challenge and scoring workflows. This supports continuity across the platform's normalization, cohorting, scoring, and feedback loop systems and maintains alignment between technical transformations performed on backend metric data and user-facing representations rendered during competition experiences.

### Model-Based Inference System

FIG. 3 illustrates an example architecture of model-based inference system 110, in accordance with various embodiments. As shown, model-based inference system 110 includes competition management system interface 302, health data acquisition system interface 304, feature engineering pipeline 306, cohort modeling engine 308, handicap modeling engine 310, training coordination module 312, model retraining scheduler 314, drift detection module 316, inference result delivery interface 318, model storage and version control system 320, training data repository 322, model output log datastore 324, and drift detection metrics datastore 326.

Competition management system interface 302 is operable to facilitate data communication between competition management system 108 and model-based inference system 110. More specifically, competition management system interface 302 is configured to receive normalized metric vectors, cohort assignment data, baseline values, and adjustment factors computed by the various modules of model-based inference system 110. Competition management system interface 302 further enables bidirectional data exchange for transmitting scoring results, leaderboard updates, and retraining triggers associated with active competitions or challenge sessions.

In various embodiments, competition management system interface 302 receives per-user health metric vectors that have been normalized, calibrated, and subjected to baseline deviation computation. These vectors may include time-aligned cohort identifiers, modality tags, device calibration weights, and intermediate scoring components generated by handicap modeling engine 310 and feature engineering pipeline 306. Competition management system interface 302 parses this input for use by competition management system 108 to update user scoring records, ranking positions, or challenge state data.

For example, competition management system interface 302 may transmit a structured payload representing a user's recent metric progression over a configurable rolling window, including heart rate, step count, and sleep efficiency—each adjusted for device calibration and population-normalized baselines. In response, competition management system 108 may apply scoring logic to update competition rankings, aggregate team scores, or trigger rule-based actions such as milestone advancement or event completion status updates. These operations enable structured synchronization between individualized scoring outputs and platform-level competition management workflows.

In certain embodiments, competition management system interface 302 supports asynchronous data exchange and event-driven communication between competition management system 108 and model-based inference system 110. For example, the interface may transmit inference results when a normalized score update exceeds a defined threshold or when drift detection metrics indicate baseline realignment is required. Payloads may be structured using JSON, protocol buffers, or other schema-conformant message formats and transmitted via authenticated API protocols such as gRPC or HTTPS. Each transmission may include model version identifiers, inference source metadata, and scoring context tags to preserve traceability and reproducibility across scoring workflows.

Competition management system interface 302 provides the link for closed-loop coordination between competition-level logic and model-based inference outputs. The interface allows the model-based inference system 110 to propagate recalibrated adjustment vectors, updated cohort assignments, and retrained model parameters to the competition management system 108 for synchronized leaderboard, reward, or eligibility computations. By enabling structured bidirectional propagation of scoring data and inference metadata, competition management system interface 302 supports machine-executed alignment of user performance data with competition-specific scoring and display logic.

Health data acquisition system interface 304 is operable to receive time-aligned, device-generated health metric data streams from health data acquisition system 104 and provide structured ingestion of the received data into model-based inference system 110 for normalization, calibration, and scoring. More specifically, health data acquisition system interface 304 retrieves physiological and behavioral signal vectors, timestamp metadata, device-type indicators, and activity modality tags for processing by feature engineering pipeline 306 and cohort modeling engine 308.

In various embodiments, health data acquisition system interface 304 is configured to ingest rolling health metric vectors including heart rate variability (HRV), sleep duration, step cadence, oxygen saturation, and respiration rate across multiple sensor modalities. The interface includes a parsing layer operable to decode transmission formats such as JSON, Protocol Buffers, or time-series binary blobs originating from wearable sensors, embedded fitness devices, or linked third-party applications. Each payload is tagged with a source device identifier and context indicator, such as the device placement (e.g., wrist, chest, ankle), measurement confidence score, and environmental sampling metadata (e.g., ambient light or temperature).

For example, health data acquisition system interface 304 may receive a continuous HRV stream recorded by a chest-worn fitness tracker during a high-effort cycling session, accompanied by cadence and speed vectors from an indoor smart bike. The interface extracts the sensor-specific records, aligns their timestamps, and forwards them to feature engineering pipeline 306, which evaluates anomaly signatures, trend inflection points, or behavior divergence to construct prompt instructions for user clarification or cohort reassignment. Similarly, the interface may identify inconsistencies in sleep pattern reporting—e.g., sharp variation in REM duration or irregular bedtime sequences—and tag such segments for refinement workflows.

In certain implementations, health data acquisition system interface 304 supports real-time stream ingestion via a persistent WebSocket or gRPC connection, and may buffer telemetry packets until fully formed activity segments are accumulated. These segments are enriched with contextual descriptors such as inferred activity type, device calibration confidence, or compliance quality score. For asynchronous ingestion, the interface may support pull-based API operations from cloud-based health data providers or schedule-based batch imports. The imported health metric vectors are serialized into intermediate structured records for downstream scoring alignment and refinement.

Health data acquisition system interface 304 may further include a tag propagation module operable to preserve inferred modality classifications—e.g., treadmill run, trail run, or elliptical—as well as population-specific biometric markers—e.g., chronotype, sleep onset latency, or heart rate reserve ratio. These values are used by cohort modeling engine 308 and feature engineering pipeline 306 to tailor cohort segmentation inputs and refine baseline alignment across comparable activity categories. The interface enables system-automated transformation of heterogeneous, noisy health data into structured, context-aware feature vectors used to generate individualized baseline comparisons and scoring inputs.

Feature engineering pipeline 306 is operable to generate structured input vectors for modeling operations performed by cohort modeling engine 308 and handicap modeling engine 310. More specifically, feature engineering pipeline 306 applies a sequence of machine-executed transformations to raw and normalized health metric records, demographic indicators, and contextual attributes to produce schema-conformant feature vectors aligned with model input specifications. The transformations performed by feature engineering pipeline 306 include vector extraction, temporal windowing, modality encoding, missing value handling, and statistical summarization across selected time periods or cohorts.

In various embodiments, feature engineering pipeline 306 ingests calibrated metric sequences (e.g., time-normalized step rate, resting heart rate, and sleep phase distributions) from health data acquisition system interface 304, user cohort assignments and demographic attributes from competition management system interface 302, and associated timestamps, device types, and environmental conditions. The pipeline segments input data into rolling windows (e.g., 7-day, 14-day, 30-day) and computes aggregate features including min/max values, mean deltas from personalized baselines, and temporal variance. For example, a user's 14-day rolling window of step rate values may be converted into a derived feature vector comprising mean steps per day, standard deviation, peak-to-average ratio, and delta-over-baseline percentage, where the baseline is computed by baseline computation module 220.

Feature engineering pipeline 306 is further operable to apply modality-aware encoding logic for inputs associated with distinct activity types (e.g., treadmill, elliptical, outdoor cycling) or situation types (e.g., intra-modality, inter-modality, and inter-category). For instance, pipeline logic may assign binary flags or categorical embeddings indicating whether a metric was captured during a standard modality or a variation, and apply normalization adjustments that preserve comparability across scoring contexts. This ensures that feature vectors accurately reflect situational equivalence for downstream model operations involving personalized scoring logic or comparative adjustments across activity contexts.

In certain embodiments, feature engineering pipeline 306 applies imputation logic to address missing or sparse time-series data using linear interpolation, last-value carryforward, demographic priors, or device-specific completion rules. The system may include a confidence scoring function that assesses completeness, temporal coverage, and outlier sensitivity of the derived feature set, and embeds the confidence level as an auxiliary input to the model. Feature engineering pipeline 306 may also encode calibration factors, device-type indicators, or historical scoring adjustments as part of the final input vector schema.

The output of feature engineering pipeline 306 comprises structured, multi-dimensional feature vectors suitable for ingestion by cohort modeling engine 308 and handicap modeling engine 310. These vectors are tagged with user identifiers, timestamp ranges, and context labels, and are stored or transmitted for predictive modeling, cohort reassignment, and personalized adjustment vector generation. In some embodiments, feature engineering pipeline 306 may include hooks for on-the-fly feature transformations, model-specific preprocessing pipelines, or data augmentation routines invoked during model retraining operations.

Cohort modeling engine 308 is operable to assign users to statistically comparable population groups based on aligned input vectors, demographic metadata, and activity modality characteristics. More specifically, cohort modeling engine 308 applies trained classification models, similarity metric evaluations, and clustering logic to feature vectors received from feature engineering pipeline 306 to determine user-to-cohort associations that serve as reference points for scoring normalization, comparative adjustments, and fairness-aware performance evaluations.

In various embodiments, cohort modeling engine 308 receives multi-dimensional input vectors including time-aligned health metrics (e.g., resting heart rate, step rate, sleep consistency), demographic attributes (e.g., age, sex, body composition indicator), device type metadata, and situational context indicators (e.g., activity type tags or inter-modality flags). These inputs are compared to cohort definition templates or prior cluster centroids stored in a model output log datastore 324. The cohort modeling engine 308 may compute similarity metrics—such as cosine distance, Euclidean distance, or Mahalanobis distance—between the user vector and each cohort vector, and select a cohort assignment based on predefined distance thresholds, probabilistic softmax distributions, or maximum likelihood scoring.

For example, cohort modeling engine 308 may evaluate whether a 47-year-old user with consistent treadmill-based activity, moderate resting heart rate variability, and recent upward deviation from a 30-day baseline belongs to a defined cluster representing middle-aged, moderately active users with improving cardiovascular trends. Assignment to this cluster would influence the selection of comparative baselines used in subsequent scoring computations, including deviation adjustment calculations and population-normalized handicap determinations. In some embodiments, the engine outputs both a primary cohort assignment and a confidence score or fallback vector for multi-cohort compatibility.

Cohort modeling engine 308 supports both fixed and dynamic cohort schemas. In static modes, the system references predefined groupings based on demographic and physiological archetypes. In adaptive modes, the engine executes unsupervised clustering (e.g., k-means, DBSCAN, Gaussian mixture models) or supervised classification (e.g., gradient-boosted trees, ensemble learners) to evolve cohort boundaries based on incoming population data or retraining triggers. The cohort definitions may be periodically refined using drift detection metrics stored in drift detection metrics datastore 326, and model retraining workflows coordinated via training coordination module 312.

In certain implementations, cohort modeling engine 308 also computes per-cohort aggregate statistics and stores them in association with each cohort identifier. These may include modality-specific baseline vectors, expected scoring distributions, demographic-normalized adjustment targets, and calibration offsets. These cohort-level aggregates are used by handicap modeling engine 310 and score computation modules to normalize individual user scores against the behavior of comparable populations. In an embodiment, cohort assignments are timestamped and versioned, allowing reproducibility of historical scoring and rollback in the event of model drift or cohort schema reconfiguration.

Handicap modeling engine 310 is operable to generate scoring adjustments based on user-specific metric trajectories, cohort-normalized benchmarks, and demographic calibration parameters. More specifically, handicap modeling engine 310 executes a layered modeling sequence to produce user-level adjustment factors that reflect improvement patterns, relative standing, and structural normalization signals, each derived from distinct vectorized input representations and stored reference distributions. These adjustments are computed for each scoring context, including intra-activity comparisons, cross-variant transitions, and inter-modality scoring scenarios.

In various embodiments, handicap modeling engine 310 receives as input: (i) individualized metric baselines—including those for core physiological biomarkers such as heart rate, sleep phases, blood pressure, or glucose variability—generated by baseline computation module 220, (ii) cohort assignment results and cohort-level aggregates from cohort modeling engine 308, and (iii) demographic indicators and device calibration data associated with the user. The engine applies one or more trained models to these inputs—such as gradient-boosted trees, ensemble learners, or weighted linear regressors—to compute distinct adjustment layers aligned with scoring dimensions referenced in competition rules and platform configuration. The engine may process each adjustment layer independently, generate confidence weights for each layer, and produce a composite handicap vector for use in downstream scoring computations.

For example, a first model may compute an improvement-based adjustment by evaluating the magnitude and consistency of a user's metric deviations from their rolling 30-day baseline, weighted by recency and volatility. A second model may generate a cohort-relative scaling factor by comparing the user's current metric vector to aggregate cohort values and assigning a percentile-based scaling output. A third model may apply a demographic calibration factor based on known age-and sex-based offsets or device-type harmonization curves derived from model output log datastore 324. These layers are then synthesized using structured inference instructions into a unified adjustment vector that informs a normalized and calibrated competition score.

In certain embodiments, handicap modeling engine 310 includes context-aware adjustment logic to account for inter-modality transitions, such as when a user performs an activity in a different modality (e.g., treadmill vs. trail) or transitions between activity types (e.g., running to cycling). This logic references stored scoring compatibility matrices or inter-modal scaling coefficients and applies transformation logic that ensures performance comparisons remain interpretable and consistent. The context-aware logic may also propagate modality flags or confidence coefficients into the output handicap vector for traceability.

Handicap modeling engine 310 may operate in batch or low-latency inference modes, depending on the cadence of competition evaluation. Adjustment vectors may be cached, versioned, and routed to inference result delivery interface 318 for integration into real-time score updates or retrospective leaderboard generation. In some implementations, the engine includes a configuration layer to activate or deactivate particular adjustment models based on regulatory requirements, fairness audits, or application-specific scoring schemes. In an embodiment, modeling operations performed by handicap modeling engine 310 are conducted using schema-conformant, machine-interpretable inputs and produce structured, traceable outputs aligned with versioned model definitions stored in model storage and version control system 320.

Training coordination module 312 is operable to manage and initiate the generation, retraining, and validation of scoring models associated with user normalization, cohort alignment, and context-specific adjustment logic. More specifically, training coordination module 312 evaluates training readiness conditions, orchestrates training workflows based on system state and performance indicators, and propagates structured training requests to model training system 114 via model training system interface 207. The coordination logic ensures that new models are trained with up-to-date data distributions and that retraining events are responsive to scoring drift or population shift conditions.

In various embodiments, training coordination module 312 monitors signals from multiple sources to determine whether a training cycle should be initiated. These sources include: (i) scoring volatility metrics computed by score computation module 224, (ii) cohort re-assignment frequencies or instability thresholds output by cohort modeling engine 308, (iii) shift indicators in modality-specific baseline vectors, and (iv) scoring consistency violations detected by drift detection module 316. Upon detecting that retraining criteria have been met—such as exceeding a configured percentile threshold of cohort overlap deviation—training coordination module 312 initiates a training job construction workflow.

The training job construction includes selecting a training schema, extracting eligible feature-label pairs from training data repository 322, and composing training payloads that include context-matched metric vectors, target labels, and model configuration metadata. The module serializes these payloads in a transport format—e.g., Parquet, protobuf, or JSONL—and transmits them to model training system 114, along with model-type indicators, training mode (initial vs. fine-tuning), and evaluation requirements. All transmitted records are version-tagged and accompanied by audit metadata to ensure reproducibility and traceability of the resulting model artifacts.

For example, training coordination module 312 may initiate a retraining job for a demographic calibration model after detecting that a new wave of wearable devices has introduced measurement variance across body composition segments. The module selects a subset of training records exhibiting representative variance, pairs these with outcome-based labels reflecting observed scoring deviations, and schedules a training cycle using a configured template for fairness-aware regression. Model training system 114 returns serialized model artifacts and evaluation metrics, which are logged by training coordination module 312 and evaluated against acceptance thresholds prior to activation.

In certain embodiments, training coordination module 312 includes version management logic for aligning downstream inference engines with the most recently validated model artifacts. This may include publishing a model activation signal to model storage and version control system 320, propagating version updates to scoring modules such as handicap modeling engine 310, and notifying drift detection module 316 of the baseline reset. Training coordination module 312 enables system-wide alignment between scoring logic and updated training distributions using structured scheduling, reproducible payload formatting, and configurable evaluation gating to ensure consistent, fair, and responsive model execution in dynamic user populations.

Model retraining scheduler 314 is operable to execute scheduled or event-triggered retraining operations associated with scoring model updates, cohort realignment, and context-aware handicap recalibration. More specifically, model retraining scheduler 314 manages a policy-driven execution calendar that initiates retraining workflows in response to system-defined intervals, performance degradation signals, or upstream control messages received from training coordination module 312. Model retraining scheduler 314 interfaces with model training system 114 and other components of the scoring pipeline to maintain continuity and version synchronization across active model instances.

In various embodiments, model retraining scheduler 314 includes configurable scheduling logic that supports both time-based (e.g., weekly, monthly) and event-based retraining strategies. Event-based triggers may include a drift detection alert received from drift detection module 316, instability thresholds reported by cohort modeling engine 308, or scoring error variance across modalities computed by handicap modeling engine 310. Time-based schedules may be aligned with competition cycles, seasonal participation trends, or wearable firmware update cadences that introduce data shifts. Retraining jobs may be categorized by type—such as full retrain, partial retrain, or calibration-only update—and prioritized according to severity metrics.

For example, model retraining scheduler 314 may be configured to perform a full retraining operation on the demographic normalization model every 90 days and to initiate intermediate calibration updates whenever scoring outputs deviate beyond a configured confidence interval across key population segments (e.g., by age bracket or device cluster). Upon trigger, model retraining scheduler 314 constructs a retraining job configuration by referencing stored templates, initiates retrieval of the corresponding training records from training data repository 322, and signals training coordination module 312 to begin model generation.

In some implementations, model retraining scheduler 314 supports parallel retraining pipelines for models associated with distinct scoring layers. For instance, a model responsible for generating individualized metric baselines may be retrained on a rolling seven-day window of user activity vectors, while a separate model for multi-user normalization may be retrained on aggregated cohort statistics spanning a broader temporal horizon. Model retraining scheduler 314 assigns each retraining task to a dedicated execution slot and logs initiation time, model identifiers, and expected completion windows to ensure system-level observability and rollback capability.

Model retraining scheduler 314 may further integrate validation gating logic to block model deployment unless performance metrics—including bias audits, stability scores, and alignment to prior cohort mappings—exceed configured acceptance thresholds. Once a model has passed evaluation, model retraining scheduler 314 publishes an activation signal to model storage and version control system 320, enabling downstream inference result delivery interface 318 to retrieve updated models for application during real-time score computation. This structured, policy-governed scheduling architecture allows for scalable, traceable retraining of handicap and cohort adjustment models across evolving user populations and device modalities.

Drift detection module 316 is operable to identify deviations in scoring behavior, cohort characteristics, and model input distributions that may indicate degradation in model performance or misalignment between current system behavior and training conditions. More specifically, drift detection module 316 executes machine-based analysis of temporal feature distributions, output score variance, and reference cohort metrics to determine whether retraining or calibration updates are warranted. Drift detection module 316 interfaces with model retraining scheduler 314 and training coordination module 312 to propagate retraining triggers and log drift indicators for downstream auditing.

In various embodiments, drift detection module 316 computes distributional statistics over normalized and calibrated metric vectors associated with recent user activity. These statistics may include rolling z-score variance, KL divergence between historical and current feature distributions, or embedding space displacement across individualized metric baselines. For example, if a rolling seven-day window of average step rate vectors shows a statistically significant shift in the centroid of a cluster associated with a specific device type or demographic group, drift detection module 316 may flag a drift event and annotate it with source metadata for retraining prioritization.

Drift detection module 316 may apply modality-specific thresholds for sensitivity to drift. For instance, sleep quality metrics derived from optical sensors may be monitored for drift at a finer granularity than cardiovascular metrics due to known device variability. Likewise, user cohorts that rely on auto-detected activity classification may exhibit higher temporal instability and trigger more frequent drift alerts. Drift detection module 316 may dynamically adjust detection thresholds based on system-wide trends or activity-specific baselines, enabling adaptive drift profiling that accounts for seasonal, demographic, or device-driven fluctuations.

In certain implementations, drift detection module 316 maintains a historical signature repository in drift detection metrics datastore 326, storing time-series vectors of cohort-wide score distributions, calibration confidence values, and metric-specific error gradients. These stored signatures serve as comparative anchors during future drift evaluations. For example, the system may compare current deviation scores against a baseline signature representing the last known stable configuration to determine whether observed changes exceed a policy-defined action threshold.

Drift detection module 316 may also perform layer-specific monitoring of downstream model outputs, including raw handicap adjustment vectors, cohort assignment probabilities, and composite scores. A detected spike in variance for score adjustment values across a previously stable cohort segment may trigger fine-tuned retraining for only that population segment. The module may annotate detected drift events with data source identifiers, user count impact, and impacted metric categories, enabling targeted scheduling of retraining tasks by model retraining scheduler 314 and structured feedback capture by training coordination module 312.

Through structured, machine-executed detection of changes in user data distributions, cohort definitions, and scoring consistency, drift detection module 316 supports the dynamic adaptation of the scoring system to evolving user behavior and measurement conditions. These operations ensure continued applicability of scoring models and preserve the integrity of context-aware competition rankings across user populations and activity modalities.

Inference result delivery interface 318 is operable to transmit model-generated scoring adjustments, cohort classifications, and calibration parameters from scoring computation components of competition management system 108 to downstream systems including user interface delivery system 112. More specifically, inference result delivery interface 318 facilitates schema-conformant communication of individualized scoring vectors, cohort label updates, and feedback confidence scores over authenticated transport protocols and structured output pipelines. The interface enables consistent propagation of inference outcomes across system components responsible for score computation, adjustment, and display.

In various embodiments, inference result delivery interface 318 formats the output of cohort modeling engine 308 and handicap modeling engine 310 into serialized, schema-conformant response payloads that include structured scoring vectors, adjustment explanations, and auxiliary metadata. These payloads may encode adjustment magnitude, scoring tier classification, modality alignment confidence, or cohort membership strength. For example, the interface may transmit a calibrated score adjustment vector indicating that a user's exertion metrics during an indoor cycling session require a +7.8 scaling factor based on recent individualized baselines and cohort-level deviation profiles. The payload may also include a confidence coefficient derived from population variance and signal completeness.

Inference result delivery interface 318 supports both push and pull communication patterns depending on the destination system. For asynchronous transmission to competition management system 108, the interface may utilize event-driven transport via message queues such as Kafka or publish-subscribe protocols. For request-based synchronization with user interface delivery system 112, the interface may expose RESTful or gRPC endpoints to return the most recent inference results for a given user session or competition cycle. The interface may also include model version identifiers and adjustment source flags in each payload to support auditability and scoring traceability.

In some implementations, inference result delivery interface 318 implements routing logic to direct output records to destination subsystems based on metric category, modality type, or scoring context. For instance, scoring vectors corresponding to time-series biometric metrics (e.g., heart rate variability, sleep duration) may be routed to scoring adjustment module 226 for integration into a weighted score computation, while context signals derived from user prompts may be routed to user interface delivery system 112 for explanatory display. Inference result delivery interface 318 may also apply data filtering, truncation, or transformation rules to tailor the delivery payload based on recipient requirements or data visibility permissions.

Inference result delivery interface 318 enables machine-based coordination of scoring decisions and user-specific adjustments across interconnected system components. By transmitting inference artifacts in structured form with traceable provenance, the interface supports the technical goal of aligning multi-layer scoring logic with downstream processing workflows and presentation layers. These machine-executed operations enable consistent and reproducible application of score transformations across diverse contexts and activity types.

Model storage and version control system 320 is operable to persist, retrieve, and manage multiple serialized versions of trained machine learning models used by the competition management system 108. More specifically, model storage and version control system 320 maintains an indexed repository of predictive model artifacts, performance metadata, training lineage information, and configuration records corresponding to cohort classification, individualized score generation, and adjustment modeling logic. The system enables consistent model access across evaluation, inference, and retraining workflows, and supports traceable rollback and reproducibility for scoring operations across distinct cohorts, devices, and activity modalities.

In various embodiments, model storage and version control system 320 stores serialized model artifacts in formats compatible with execution frameworks used by cohort modeling engine 308 and handicap modeling engine 310, including ONNX, TensorFlow SavedModel, or PyTorch TorchScript formats. Each stored model instance is associated with a version identifier, training snapshot hash, configuration manifest, and performance record including metrics such as scoring accuracy, calibration error, fairness indices, and modality alignment coefficients. For example, a model trained to adjust scoring behavior for age-specific cohorts in cycling activities may be tagged with a unique identifier (e.g., score_adjust_cycling_age_v3.1), associated training timestamp, and metadata indicating its use with indoor bike telemetry normalized to five-minute intervals.

Model storage and version control system 320 enables atomic writes and transactional updates to ensure consistency across dependent systems accessing models for real-time inference or batch scoring. The system may expose programmatic access interfaces to retrieve the latest stable model for a given scoring context, validate model signatures, or enumerate historical versions for retrospective analysis. For example, model retraining scheduler 314 may request a prior model version to compare inference drift between scoring periods or to retrain a derivative model using historical configuration templates.

In some implementations, model storage and version control system 320 includes a branching mechanism that allows multiple experimental model variants to coexist within a structured namespace. These branches may correspond to different demographic normalization strategies, feature encoding schemas, or competition formats. Model lineage tracking is supported via dependency graphs that link each model version to its corresponding training dataset (e.g., from training data repository 322), hyperparameter configuration, and scoring baseline. The system may further implement access controls and audit logging to enforce integrity and compliance during model retrieval and deployment.

By enabling structured model versioning and reproducible storage of machine-executed scoring logic, model storage and version control system 320 supports the technical objective of consistent and auditable competition outcomes across scoring modalities. These capabilities address the technical problem of managing evolving model behavior in a dynamic population while supporting differentiated scoring across varying activities, demographic profiles, and device ecosystems.

Training data repository 322 is operable to store schema-conformant training datasets that support machine-executed modeling workflows for context-aware scoring, individualized baseline generation, and cohort-level adjustment modeling. More specifically, training data repository 322 persists structured feature vectors, adjustment labels, device metadata, and modality-aligned scoring outcomes generated by upstream components such as feature engineering pipeline 306, cohort modeling engine 308, and handicap modeling engine 310. The stored datasets support supervised, semi-supervised, and reinforcement-based training of models used throughout competition management system 108 and associated inference workflows.

In various embodiments, training data repository 322 stores time-indexed user metric vectors derived from raw health telemetry and normalized by health data acquisition system interface 304, with features including calibrated biometric sequences, modality tags, cohort indicators, and baseline deviation values. For example, a training record may include a rolling seven-day vector of resting heart rate deltas, normalized to a modality-specific baseline vector for indoor cycling, along with a demographic vector (e.g., age, biological sex, device model), a scoring label reflecting prior adjustment logic, and a timestamped activity tag. These records enable training of models that support both modality-specific and cohort-normalized scoring across heterogeneous user populations.

Training data repository 322 supports multi-resolution partitioning schemes that enable efficient retrieval of training data based on cohort category, activity modality, demographic segment, or scoring context. Partitioning may be implemented using temporal shards, cohort membership indices, or device-type filters to optimize batch construction for model training or retraining cycles. For example, model retraining scheduler 314 may request only those training records associated with cohorts showing drift in recent scoring periods, enabling targeted refresh of the adjustment logic without reprocessing the full dataset.

In some implementations, training data repository 322 maintains audit metadata alongside each training record, including lineage hashes, transformation identifiers, and scoring version tags. These metadata fields ensure reproducibility of model behavior and traceability of training input sources. For example, a record tagged with transformation ID norm_v2.3, cohort version C15, and scoring configuration score_cfg_fairness_1.1 allows downstream systems to reproduce or validate training conditions during model evaluation. The repository may also store holdout and validation subsets for cross-validation and fairness testing, including scoring parity metrics stratified by modality or demographic group.

Training data repository 322 enables the computing system to transform unstructured metric data into structured, reproducible training records that support context-specific and demographically aligned scoring models. These capabilities address the technical challenge of maintaining scalable, traceable model training across evolving populations, activity contexts, and device ecosystems, and ensure that model behavior reflects consistent handling of individualized improvement, modality transitions, and cohort-specific normalization.

Model output log datastore 324 is operable to persist structured logs of inference results, score adjustment outputs, and baseline alignment metadata generated by models executed within cohort modeling engine 308 and handicap modeling engine 310. More specifically, model output log datastore 324 stores system-executed scoring outputs, model version identifiers, feature attribution scores, and user-specific adjustment records for downstream analysis, fairness auditing, scoring traceability, and performance monitoring across scoring contexts and activity modalities.

In various embodiments, model output log datastore 324 captures model-generated scoring outputs associated with user records and tagged by scoring context, model version, and adjustment source. For example, an entry in model output log datastore 324 may include a user ID, a normalized score vector aligned to a modality-specific baseline for rowing, a demographic adjustment offset, and a model-generated cohort classification label. Each entry includes the inference timestamp, model version hash (e.g., hmd_v4.2), and contextual metadata such as the applied normalization schema, transformation rules, or modality tags.

Model output log datastore 324 supports fine-grained auditability by storing both the input vector representation used at inference time and the corresponding output results, enabling deterministic replay of scoring operations and structured validation of score integrity. For example, if a scoring adjustment engine produces an updated competition score based on new calibration coefficients, model output log datastore 324 can be queried to retrieve the original feature vector, applied model parameters, and scoring outcome for that time window. These capabilities support longitudinal tracking of model outputs and alignment with external competition ranking logic.

In certain implementations, model output log datastore 324 supports stratified storage and retrieval of inference records based on user cohort, scoring context, or detected data drift markers. For example, output logs may be indexed by situation type (e.g., transitions across different activity modalities or scoring contexts), allowing the system to isolate model performance variations when comparing modality-conformant transitions (e.g., treadmill to outdoor run) or evaluating scoring consistency for user populations with different device types or demographic profiles. These stratifications enable targeted validation of fairness-preserving behavior across a distributed scoring architecture.

Model output log datastore 324 enables the computing system to maintain a persistent, queryable record of model-executed scoring outcomes, including contextual tags, calibration metadata, and input-output pairings that facilitate evaluation and system-wide reproducibility. This structured audit trail supports technical objectives related to traceable model performance, dynamic version tracking, and equitable scoring analysis across user groups, thereby addressing system-level requirements for transparent, accountable application of score-generating machine learning models.

Drift detection metrics datastore 326 is operable to store computed indicators, statistical divergence scores, and context-specific performance metrics used to monitor inference stability, model generalization, and population consistency across cohorts and activity modalities. More specifically, drift detection metrics datastore 326 persistently records machine-generated outputs from drift detection module 316, including temporal baseline deviations, scoring distribution shifts, modality-level entropy metrics, and demographic parity trends associated with deployed model behavior.

In various embodiments, drift detection metrics datastore 326 includes version-aligned metrics that quantify the divergence between training and inference distributions, enabling structured comparison of model performance over time. For example, the datastore may store population-wide score variance comparisons, KL-divergence between recent and historical feature distributions, or a moving-window z-score trend for an individualized metric baseline associated with a particular scoring context. These records may be linked to cohort modeling engine 308 outputs to enable correlation analysis between assignment drift and observed model degradation.

Drift detection metrics datastore 326 supports indexed access to drift indicators by model version, cohort ID, activity modality, device category, or demographic group. For example, in response to a model retraining scheduler trigger, drift detection module 316 may compute a modality-constrained scoring shift for users transitioning between rowing and cycling contexts, and store per-cohort Wasserstein distances indicating score instability across those contexts. The datastore may flag scoring asymmetries exceeding defined thresholds, allowing downstream validation workflows to prioritize segments with high variance or structural imbalance.

In certain implementations, drift detection metrics datastore 326 includes historical rollups of scoring trends aggregated by week, cohort, or modality group, enabling multi-scale tracking of model health and alignment with individualized baselines. These metrics may be visualized via system dashboards, exported to training coordination module 312 for retraining priority ranking, or compared against historical calibration consistency thresholds to determine whether drift exceeds system-defined tolerance levels. In some embodiments, drift detection metrics datastore 326 stores both raw drift indicators and post-processed risk scores used by automated retraining pipelines to allocate computational resources based on drift severity.

Drift detection metrics datastore 326 enables the system to maintain a persistent and structured representation of machine-generated statistical monitoring signals, computed across personalized, demographic, and modality-specific partitions of the health metric population. This supports technical operations involving automatic detection of inference instability, model degradation, and score normalization decay—facilitating reproducible tracking of divergence trends and enabling system-initiated mitigation actions through retraining, cohort reassignment, or calibration parameter revision.

User preference datastore 330 is operable to persist individualized scoring parameters, including rolling metric baselines, assigned cohort identifiers, context-specific scoring configurations, and preference tags associated with each user. More specifically, user preference datastore 330 stores baseline values generated by feature engineering pipeline 306, cohort assignments computed by cohort modeling engine 308, and configuration flags used by handicap modeling engine 310 to apply personalized scoring logic. In some embodiments, user preference datastore 330 includes versioned records of per-metric scoring configurations, such as metric directionality (e.g., “lower is better”), modality-specific scaling factors, or flags indicating whether a given biomarker should be included in competition-level scoring. The datastore supports efficient lookup and traceability for inference workflows executed by handicap modeling engine 310 and inference result delivery interface 318.

### Training Pipeline

FIG. 4 illustrates an example machine learning training and evaluation pipeline for generating and updating cohort-normalized scoring models, in accordance with various embodiments. As shown, the system processes a normalized training dataset 402, which includes schema-conformant metric vectors, cohort assignment metadata, device calibration values, and individualized baseline vectors generated from time-series health data acquired and processed as described in FIGS. 1-3. These training records may include labeled handicap outcomes and adjustment factors derived from retrospective user comparisons within matched cohorts. In some embodiments, labels are assigned using a combination of model-inferred scoring outcomes and expert-reviewed challenge results based on longitudinal competition data.

Normalized training dataset 402 is ingested by model training module 404, which is operable to generate trained model(s) 406. More specifically, model training module 404 executes supervised and semi-supervised learning routines to train machine learning models configured to generate individualized handicap predictions. The models may be trained on input features that include calibrated health metrics, modality-specific baseline vectors, contextual cohort indicators, and scoring labels representing observed or derived adjustment targets. The training process may apply regularization techniques to preserve generalization across diverse user populations and may incorporate federated or privacy-preserving learning frameworks when operating across distributed datasets.

The resulting trained model(s) 406 may include neural networks, gradient boosting ensembles, or transformer-based architectures configured to output normalized scoring parameters aligned to scoring contexts described herein. These include adjustments within a consistent activity type, across variations of a baseline activity, and across distinct activity types. In certain implementations, a plurality of trained models may be maintained for different demographic segments, device ecosystems, or scoring modalities. The training system may additionally project user metric vectors into a latent scoring space using learned embedding layers, enabling vector distance-based calibration and similarity-aware adjustments.

Trained handicap model(s) 406 are evaluated using model evaluation module 410, which receives holdout evaluation dataset 408 comprising previously unseen user records. These records include normalized health metric vectors, individualized baseline values, demographic metadata, and device profile parameters not included in training dataset 402. Model evaluation module 410 is operable to calculate quantitative performance metrics, such as accuracy of predicted adjustment factors, fairness distribution across segmented cohorts, percentile error rates, and scoring calibration consistency across activity transitions. Based on these metrics, model evaluation module 410 may trigger retraining workflows, flag drift conditions, or enable model selection routines that promote the deployment of the most contextually stable and population-consistent handicap models. In certain embodiments, model evaluation module 410 applies bootstrap-based variance estimation or rolling error bounds to assess generalization robustness across score percentiles.

Upon successful evaluation, trained handicap model(s) 406 are stored for production use and made available to handicap prediction module 414. Handicap prediction module 414 is operable to apply trained handicap model(s) 406 to new user data vector 412. More specifically, handicap prediction module 414 receives calibrated health metric vectors, contextual cohort metadata, and modality-specific baseline values for a given user, and computes a final handicap score and adjustment factors. These outputs may be used by downstream systems to generate normalized competition scores, produce cohort-aligned challenge outcomes, or update the user's longitudinal profile in the scoring datastore.

In certain embodiments, model training module 404 or handicap prediction module 414 applies model-specific inference logic that accounts for cohort dynamics, calibration drift, or temporal variance in user performance. For example, time-weighted cohort baselines may be computed for older users transitioning between walking and stationary cycling modalities, and corresponding adjustment factors may be predicted to preserve interpretability and fairness across activity contexts. These operations represent machine-executed transformations over multi-modal time-series data and demonstrate integration of structured training workflows with adaptive scoring logic under the control of a dynamically evolving model architecture. Trained handicap model(s) 406 may be stored as version-controlled artifacts linked to training parameters, data schema hashes, and model lineage metadata to support rollback, reproducibility, and comparative evaluation across model generations.

### Process for Model Training

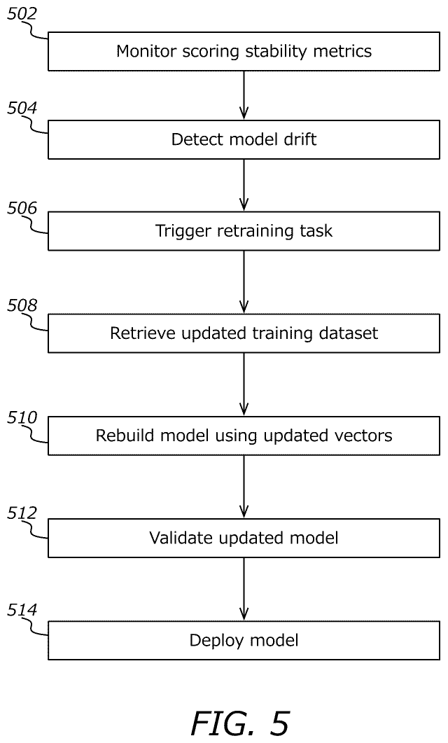

FIG. 5 illustrates an example process for monitoring model stability, detecting scoring drift, and triggering adaptive retraining workflows, in accordance with various embodiments. The process steps shown may be performed by components of the model training system 114, including training coordination module 312, model retraining scheduler 314, drift detection module 316, and model evaluation module 410, as described in FIGS. 1-4.

In various embodiments, the steps are implemented using processor-executable instructions stored in non-transitory memory and executed by one or more computing systems configured to monitor performance metrics, detect baseline or cohort degradation, and initiate model retraining and deployment. The operations may be executed in batch or streaming mode, depending on the system configuration, and may be implemented using distributed computing infrastructure or specialized model orchestration frameworks. Additional steps, fewer steps, or reordered steps may be performed within the scope of the invention, depending on the retraining cadence, model architecture, or scoring requirements.

In step 502, scoring stability metrics are monitored over time using time-series scoring outputs generated by inference system 110. Scoring stability metrics include intra-user score variance, inter-cohort score spread, percentile error across demographic bins, and temporal prediction confidence. Time-series scoring outputs include longitudinal handicap scores, normalized context-specific outcomes, and associated calibration metadata. For example, drift detection module 316 is operable to track scoring trends across repeated activities within the same modality, detect shifts in cohort placement over time, and quantify prediction certainty across demographic segments. Stability metrics may be collected for each user across multiple scoring contexts and activity modalities, and may include metadata aligned to modality-specific baseline vectors, historical adjustment factors, and scoring context tags. These scoring stability metrics are logged and stored in drift detection metrics datastore 326 for further analysis in step 504.

For example, drift detection module 316 may generate a rolling summary of handicap score variance for a given user over a seven-day period, segmented by activity type and device profile. If the system observes that score distributions deviate significantly from cohort means or exhibit increased fluctuation during transitions between modalities (e.g., walking to elliptical), those sequences may be flagged for further evaluation. Similarly, population-level stability metrics may include cohort alignment scores—representing the consistency of percentile placements within each cohort—or calibration curve divergence metrics computed across model versions.

In certain embodiments, drift detection module 316 applies statistical aggregation and smoothing techniques (e.g., exponentially weighted moving averages, interquartile filtering) to suppress noise and isolate systemic deviations. The resulting scoring stability metrics are timestamped, user-aligned, and stored in drift detection metrics datastore 326 for downstream analysis. These stored metrics serve as the basis for detecting concept drift, cohort boundary instability, or calibration decay in subsequent processing stages.

In step 504, model drift is detected based on observed deviations in scoring performance and cohort alignment patterns. Drift detection module 316 is operable to analyze scoring stability metrics retrieved from drift detection metrics datastore 326 and compare these metrics against predefined thresholds, baselines, or expected distributions. Model drift may be identified based on indicators including rising intra-cohort scoring variance, declining prediction confidence for previously stable activity contexts, or divergence between expected and observed adjustment factors across new user populations. For example, if percentile-normalized handicap scores for a specific demographic cohort begin to cluster outside the expected range across multiple activity types, the system may infer a misalignment between current model parameters and updated cohort baselines. In certain embodiments, scoring consistency is validated using modality-specific baseline vectors, and statistical tests are applied to detect distributional shifts in calibrated health metrics or adjustment scores. These operations represent machine-executed comparisons over multi-dimensional feature spaces and are used to trigger model retraining workflows when prediction quality or fairness metrics degrade beyond acceptable thresholds.

In step 506, a retraining task is triggered in response to the detection of model drift or performance degradation. Drift detection module 316 is operable to publish a retraining event signal to training coordination module 312, which initiates a structured training cycle based on the type and scope of the detected drift. For example, if accuracy degradation is localized to a particular activity modality or demographic cohort, the system may trigger a partial retraining sequence using a filtered training dataset aligned to that scope. In other scenarios, a full retraining workflow may be executed using updated cohort metadata, revised baseline vectors, and longitudinal performance records retrieved from model output log datastore 324 and training data repository 322. Training coordination module 312 may serialize a job definition specifying model architecture, hyperparameter constraints, inclusion criteria for training samples, and performance benchmarks for model acceptance. In certain embodiments, the retraining trigger includes metadata describing drift type, source signal, affected scoring contexts, and prior model version identifiers, enabling downstream components to log model lineage and orchestrate deployment gating. These operations allow the computing system to respond to statistical degradation or fairness imbalance by automatically initiating model retraining pipelines with context-specific input constraints and validation targets.

In step 508, training coordination module 312 is operable to retrieve an updated training dataset from training data repository 322 in response to a retraining trigger. The retrieved dataset includes newly ingested and calibrated health metric vectors, revised cohort labels, updated modality-specific baseline vectors, and performance context tags aligned to recently detected scoring conditions. More specifically, the updated dataset may be filtered to include user records from affected cohorts, demographic segments, or activity modalities where drift or fairness imbalance was detected, as logged by drift detection module 316 and model output log datastore 324.

For example, if inter-cohort score variance increased among users transitioning from strength training to HIIT sessions, the training coordination module 312 may extract metric vectors corresponding to those transitions, including individual baseline deviations, device calibration profiles, and prior score adjustments. Metadata specifying temporal alignment, cohort configuration versioning, and scoring stability indicators is appended to the training batch to support supervised model learning and post-training validation.

In certain embodiments, the updated training dataset includes annotations derived from semi-structured user feedback or engagement events, which may be interpreted by LLM-based augmentation services to extract retraining signals. These annotations may reflect score mismatch complaints, unexpected challenge results, or shifts in perceived fairness—transformed into structured labels using prompt-based natural language parsing pipelines. This allows the retraining dataset to include both numerical and semantically inferred data elements, contributing to downstream model robustness in context-sensitive scoring applications.

In step 510, model training module 404 is operable to rebuild the scoring model using the updated training dataset retrieved in step 508. More specifically, model training module 404 executes a supervised or semi-supervised learning pipeline that incorporates the most recent metric vectors, individualized baseline sequences, and cohort segmentation metadata. The updated data is aligned to the latest schema and scoring context configuration, ensuring that model inputs reflect current device characteristics, demographic distributions, and scoring modalities.

Model training module 404 processes the updated dataset using training routines that preserve architectural continuity or apply updated hyperparameters, depending on the retraining trigger classification. For example, if scoring inconsistency was traced to calibration drift in optical heart rate sensors across younger users, the system may initiate a partial retraining operation that reuses embedding layers while updating regression heads tuned to that subgroup. Alternatively, if cohort boundaries shifted due to demographic rebalancing, model training module 404 may execute full retraining with revised cohort encodings.

The model architecture may include neural network ensembles, attention-weighted scoring heads, or transformer-based encoders trained to generate adjustment factors and normalized scores aligned to scoring contexts. Regularization techniques such as dropout, weight decay, or elastic net constraints may be applied to prevent overfitting to short-term score fluctuations. Training metadata, including training timestamp, cohort version, baseline window parameters, and label confidence weights, may be logged to model output log datastore 324 for traceability.

In certain implementations, the training process incorporates scenario-specific replay buffers or synthetic augmentation techniques. For instance, time-warped versions of previous user sequences may be injected to enforce temporal robustness, or LLM-generated user narratives may be converted into feature vectors and adjustment labels to expand underrepresented context configurations. These machine-executed augmentations contribute to increased model generalizability across scoring scenarios with limited historical representation.

In step 512, the updated scoring model is validated using holdout evaluation datasets and performance benchmarks to determine its suitability for deployment. Model evaluation module 410 is operable to execute a structured evaluation routine using previously unseen user data vectors that include calibrated health metrics, demographic features, modality-specific baseline vectors, and corresponding outcome labels or derived adjustment targets.

More specifically, model evaluation module 410 computes quantitative metrics such as prediction accuracy, calibration alignment, inter-cohort fairness distribution, percentile-based scoring error, and scoring stability across adjacent context transitions. For example, the system may calculate whether predicted adjustment factors preserve ranking consistency among users performing the same activity across distinct age groups, or whether fairness ratios between high-and low-baseline users remain within configured tolerances.

Model evaluation module 410 may also compute drift-aware comparison metrics between the updated model and the previously deployed version. In certain embodiments, delta thresholds are applied to assess whether the scoring variance, demographic parity gap, or calibration error exceeds acceptable bounds. Where multiple trained models are available, model evaluation module 410 selects the optimal candidate based on a composite evaluation score computed from weighted performance indicators.

In some implementations, model evaluation includes simulated deployment runs using historical competition sequences replayed through the updated model. These synthetic evaluations may expose score oscillation, sensitivity to edge-case inputs, or unintended overfitting to recent activity clusters. Additionally, the validation routine may incorporate confidence-based gating, where low-certainty predictions are flagged for further review or routed through ensemble agreement mechanisms.

Evaluation results, including metric outputs, score histograms, model selection rationale, and timestamped validation metadata, may be logged in model output log datastore 324 and linked to the versioned model entry in model storage and version control system 320. This structured validation framework supports traceable, auditable decision-making for subsequent deployment steps.

In step 514, the validated scoring model is deployed to inference system 110 for use in generating cohort-normalized competition scores and context-aware adjustment factors. Deployment is coordinated by training coordination module 312, which interacts with model storage and version control system 320 to retrieve the selected model artifact, verify its integrity, and publish the model to a designated scoring environment.

More specifically, training coordination module 312 performs a versioned release process that registers the updated model in a deployment manifest, links corresponding validation results stored in model output log datastore 324, and propagates the model to the active inference container or runtime node associated with the competition management system interface 302. The deployment manifest may encode model signature, configuration parameters, cohort mappings, applicable baseline schema versions, and scoring context tags used for runtime selection.

In certain embodiments, the deployment process includes warm-start procedures to preload embedding weights or scoring logic into memory-efficient data structures, enabling low-latency inference on streaming user inputs. The system may also support staggered rollout or A/B deployment strategies, where a percentage of incoming user data is routed to the updated model while legacy scoring remains active for comparison.

To support traceability and rollback, model deployment metadata—including deployment time, configuration hashes, and upstream validation results—are recorded in model output log datastore 324 and indexed against cohort identifiers or activity contexts. In some implementations, the system may generate deployment alerts or confirmations to administrative dashboards, including real-time telemetry of initial scoring outcomes under the new model.

Upon deployment, inference system 110 applies the model to new user data vectors to compute individualized scoring outputs in accordance with the population-normalized handicap model, updated baseline vectors, and activity context tags, enabling downstream modules such as score computation module 224 and challenge orchestration module 228 to generate context-consistent competition outcomes.

### Process for Computing a Normalized Score

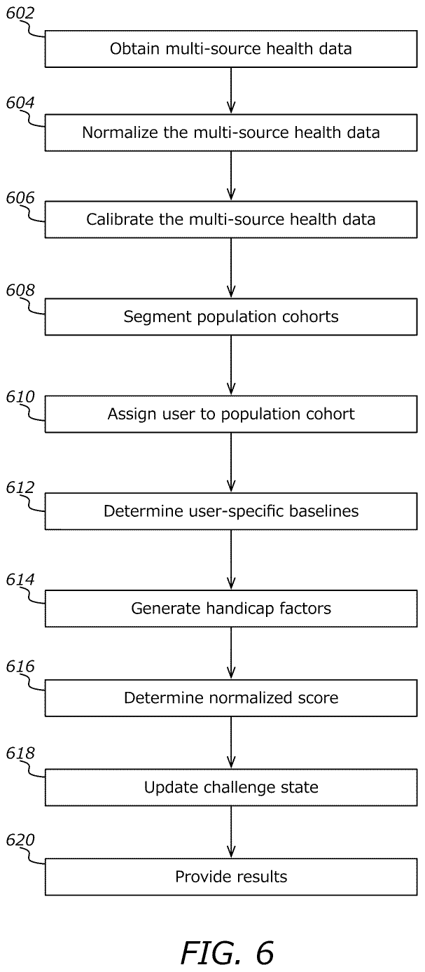

FIG. 6 illustrates an example process for computing a normalized score using cohort-based handicap modeling in accordance with various embodiments. The method steps depicted in FIG. 6 may be implemented as processor-executable instructions executed by inference system 110 and associated modeling subsystems of the system architecture shown in FIG. 1, including but not limited to inference system 110 and associated modeling subsystems. In various embodiments, the process may be executed continuously or periodically, and supports configuration based on activity modality, device ecosystem, or competition format.

In step 602, multi-source health data is obtained. Multi-source health data can include biomarkers such as physiological, behavioral, and contextual measurements recorded over time, such as heart rate, steps, calories, sleep duration, respiration rate, or perceived exertion. The multi-source health data can be obtained from a plurality of health tracking sources communicatively coupled to the system through health data acquisition system interface 304 and external system integration gateway interface 204. The obtained data comprises unstructured or semi-structured health metric records associated with a user, with each record including heterogeneous metric values collected across disparate device types (e.g., smartwatches, chest straps), sensor modalities (e.g., optical, accelerometric, electrodermal), and software ecosystems (e.g., fitness platforms, wellness applications).

In certain embodiments, multi-source health data further includes higher-order biomarkers derived from specialized sensors or external lab integrations, such as blood glucose concentration, blood pressure, oxygen saturation, cortisol levels, and sleep phase scoring (e.g., REM, deep, or light sleep durations). These biomarkers may be received from biometric monitoring platforms, integrated through third-party APIs, or inferred through data fusion models applied to multi-modal inputs. For example, a continuous glucose monitor (CGM) may transmit rolling glucose readings in mg/dL, while a connected sleep tracker may report REM sleep percentage and total restorative sleep duration in 5-minute epochs.

The health tracking sources can include commercial wearables, mobile health applications, biometric sensor hubs, and third-party fitness ecosystems capable of exporting or streaming user activity data. For example, a smartwatch may upload heart rate and movement data via Bluetooth, a mobile app may provide self-reported sleep quality scores via RESTful API, and a gym-based cardio machine may export exercise session summaries over FTP or cloud sync. The system associates each record with its device identifier, sampling resolution, sensor origin, and time of capture to establish traceability and input context.

The received records may be temporally misaligned and structurally inconsistent, with varying field labels, units of measure, and completeness. As such, the system queues these records for ingestion and tagging, ensuring that every input sample is linked to a valid user profile and system session. This enables subsequent normalization of heterogeneous metric data across different devices and activity types while maintaining source fidelity for calibration and scoring processes.

In step 604, the multi-source health data is normalized to produce a set of standardized metric values. Normalization includes converting unstructured or semi-structured health metric records into a schema-conformant format suitable for downstream calibration, scoring, and model-based processing. This normalization process is executed by feature engineering pipeline 306 and involves transforming metric records collected from heterogeneous device types and activity modalities into aligned, machine-readable vectors. These schema-conformant vectors form the structured input used by feature engineering pipeline 306 to produce time-windowed baselines and scoring inputs described in subsequent steps.

The normalization process includes unit conversion (e.g., kilocalories to joules, miles to kilometers), timestamp harmonization (e.g., aligning sampling intervals across devices), and schema mapping (e.g., transforming vendor-specific field names into platform-defined metric identifiers). For example, a heart rate field labeled bpm_reading in one device payload may be remapped to a standardized heart_rate field, while ensuring that corresponding time-of-measurement fields are adjusted to match the system's canonical timeline using UTC offsets or sensor clock drift corrections.

In certain embodiments, data field mappings are derived from source-specific schemas stored in a metadata repository, and the normalization process uses rule-based logic to align source values to platform-defined metric definitions. The output of this process is a temporally indexed vector of normalized metric values, with each element tagged with device provenance, modality context, and metric type identifier. These normalized vectors enable consistent processing across users and systems, regardless of device or platform heterogeneity, and support reproducible downstream scoring computations based on user-specific and cohort-based reference parameters. In certain implementations, these normalized metric vectors are expressed as machine-interpretable records using an internal schema definition language (SDL) compatible with downstream model inputs.

In step 606, the normalized metric values are calibrated using device profile data corresponding to the health tracking sources from which the original metric records were obtained. Calibration is performed to correct for known measurement deviations, sampling biases, and device-specific idiosyncrasies that can affect the interpretability of metric values across disparate hardware or software ecosystems. This calibration process is executed by feature engineering pipeline 306 in coordination with calibration profiles stored in item attribute datastore 332.

Device profile data includes empirically derived correction coefficients, accuracy bands, and error model parameters associated with individual sensor models or device classes. For example, if a wearable device model is known to consistently underreport resting heart rate by 5-7%, a corresponding correction coefficient may be retrieved and applied to the normalized metric values from that device. Similarly, accelerometer-derived step counts from a wrist-based tracker may be calibrated using a step overcount suppression factor derived from gait analysis studies or internal benchmarking datasets.

In certain embodiments, calibration also incorporates context-specific adjustments based on device placement (e.g., wrist vs. hip), sensor fusion configuration (e.g., GPS+IMU vs. GPS-only), or operating environment (e.g., indoor treadmill vs. outdoor terrain). The calibration process applies these corrections in a schema-conformant and time-indexed manner, updating each metric vector element with a calibrated value that reflects both the original sensor reading and the correction model retrieved from the device profile data.

The calibrated metric values are stored as feature vectors annotated with correction metadata, and serve as the input for baseline generation, cohort assignment, and scoring processes in subsequent steps. These calibrated outputs support technically robust comparisons across users with different devices, ensure compatibility with cohort-based reference parameters, and enable reproducible model-based adjustment calculations.

In step 608, a population of users is segmented into a plurality of population cohorts based on demographic data and device metadata associated with each user. This segmentation process is performed by cohort modeling engine 308, which applies machine-executed logic to group users who share statistically relevant characteristics that impact health metric interpretation or scoring comparability. The resulting population cohorts serve as structured references for baseline modeling, expected value computation, and context-specific scoring normalization.

The demographic data may include features such as age, sex, weight, height, geographic location, and self-reported health conditions. Device metadata may include device model, firmware version, sensor type (e.g., optical heart rate sensor vs. ECG), sampling frequency, and platform ecosystem (e.g., Apple HealthKit vs. Google Fit). Together, these attributes define a user profile vector that serves as input to a segmentation algorithm.

In various embodiments, cohort modeling engine 308 applies unsupervised clustering techniques—such as k-means, DBSCAN, or Gaussian mixture modeling—to the user profile vectors. The number of cohorts, cluster centroids, and assignment thresholds may be computed dynamically based on data density and cohort balance objectives. For example, users aged 50-60 using wrist-based photoplethysmography (PPG) sensors with low sampling rates may be grouped into a cohort that exhibits different baseline heart rate variability (HRV) characteristics compared to younger users using chest-strap ECG monitors.

Each population cohort is defined by a centroid vector and metadata tags that describe the defining attributes of the group. These cohort definitions are stored in the user preference datastore 330 and indexed for retrieval during cohort assignment and scoring workflows. The cohort segmentation logic enables the computing system to support population-normalized modeling techniques, apply consistent reference parameters, and maintain fairness across users with diverse demographic and device configurations. This segmentation step forms the structural foundation for deviation computations and adjustment vector generation in subsequent processing stages.

In step 610, the system assigns the user to a selected population cohort of the plurality of population cohorts generated in step 608. This assignment is performed by cohort modeling engine 308 using the user's demographic data and device metadata as matching criteria against the precomputed cohort centroids and assignment rules. The result is a structured mapping between the individual user and a statistically similar group of users, enabling consistent application of cohort-specific reference parameters in subsequent scoring logic.

The user's demographic data and device metadata are first transformed into a user profile vector using the same schema and feature scaling logic employed during cohort segmentation. Cohort modeling engine 308 retrieves a set of candidate cohort centroids and computes a distance metric between the user profile vector and each centroid. In certain embodiments, the distance metric is based on Euclidean distance, Mahalanobis distance, or cosine similarity, depending on the distribution and correlation of input features.

For example, a user with the following profile: age 55, female, using a wearable with a known ±5 BPM HR bias and 1 Hz sampling rate, may be matched to a cohort where the centroid profile includes users aged 50-60 using similar devices with comparable accuracy profiles. The selected cohort may be identified as having stable intra-cohort baselines for metrics such as resting heart rate, step count, and sleep efficiency under matched conditions.

Assignment confidence thresholds or soft clustering logic may be used to determine whether the user falls squarely within a cluster boundary or straddles multiple clusters. In such cases, weighted cohort blending or probabilistic scoring strategies may be invoked in downstream steps. Once assigned, the user's cohort ID is stored in user preference datastore 330 and linked to the user's longitudinal profile for use in model-based inference, adjustment vector generation, and normalization routines. This step ensures cohort-specific scoring parameters are consistently applied, enabling machine-executed transformations of raw health data into normalized scores that reflect both individual baselines and population-level context.

In step 612, a baseline value is generated for each health metric associated with the user based on a rolling time window applied to the user's calibrated metric values. This baseline computation is performed by feature engineering pipeline 306 using health data that has been normalized and calibrated in steps 604 and 606, respectively. The baseline value represents a time-aware individualized reference point for each metric, enabling per-user contextualization during subsequent scoring.

More specifically, feature engineering pipeline 306 retrieves a time-series of calibrated metric values for each metric dimension (e.g., heart rate, resting heart rate, steps, HRV) and applies a rolling window defined by a configurable recency interval (e.g., 7-day, 14-day, or 30-day windows). Within the rolling window, the system applies a weighted moving average function in which weights decrease with recency, thereby emphasizing more recent data while maintaining statistical smoothing. This function accounts for short-term variations in behavior while preserving stable trend lines used in baseline formation.

For example, a user's resting heart rate over the past 14 days may range from 57 BPM to 62 BPM. Applying a recency-weighted moving average may yield a baseline of 60.2 BPM, which reflects recent trends while smoothing out short-term fluctuations. Baselines for other metrics such as steps or HRV may be computed using different weighting functions depending on metric volatility or modality sensitivity.

In some implementations, baseline values may incorporate modality-specific partitioning, such that separate baselines are computed per activity type or device context. For instance, if a user alternates between outdoor runs and treadmill runs, the system may compute two separate baselines for step cadence or distance to enable context-aware scoring downstream.

The computed baselines are stored in user preference datastore 330 and linked to the user's versioned scoring profile record therein. These individualized baseline values serve as the foundation for deviation modeling and adjustment vector generation in subsequent steps, anchoring the scoring transformation to a machine-learned, data-driven representation of the user's typical performance under observed conditions. In certain embodiments, individualized baselines for advanced biomarkers (e.g., blood pressure, REM sleep duration, or glucose variability) are also computed and stored, enabling downstream scoring logic to incorporate more granular indicators of physiological stability or variation. These additional dimensions may be used to refine context-aware adjustment logic or support extended competition formats involving metabolic or recovery-based performance metrics. These enriched baseline representations ensure reproducibility of adjustment vector generation and support schema-conformant feature inputs for the layered modeling operations described in FIG. 3 and FIG. 5.

Once cohort and baseline values are available, the system generates scoring adjustment factors. In step 614, one or more handicap factors are generated based on the user's individualized metric baselines, population cohort characteristics, and demographic calibration parameters. This operation is performed by handicap modeling engine 310 using a layered modeling sequence configured to produce adjustment outputs aligned with multiple scoring dimensions. The resulting handicap factors form a structured adjustment vector applied during score normalization and comparative evaluation routines in subsequent steps.

Handicap modeling engine 310 retrieves as input: (i) the individualized baseline values generated in step 612 and stored in user preference datastore 330, (ii) the cohort-level aggregates and expected metric distributions associated with the user's cohort assignment from step 610, and (iii) demographic and device calibration data associated with the user's profile. Each input vector is encoded in a schema-conformant format and processed through one or more trained model layers, each configured to compute a distinct adjustment dimension based on structured inference logic. These layers may include improvement tracking models, population-relative scaling models, and demographic normalization models, each trained using historical scoring outcomes and cohort-evolution datasets stored in model output log datastore 324.

For example, a first model layer may evaluate deviation magnitude and volatility between a user's recent rolling baseline (e.g., resting heart rate) and current observation to produce a dynamic improvement factor. A second model may compute a percentile-based adjustment factor by comparing the user's normalized metric vector against cohort-wide scoring centroids, applying scaling logic that reflects relative standing within a statistically similar population. A third layer may apply demographic normalization coefficients using linear offset or multiplicative scaling to account for age-or sex-based performance differentials observed across similar device types or activity contexts. Each factor is computed independently, assigned a confidence score, and tagged with a version identifier associated with the underlying model parameters retrieved from model storage and version control system 320.

The handicap factors may be synthesized into a composite adjustment vector using structured inference instructions that preserve independence of scoring dimensions while enabling unified application within normalized score computation. For instance, in certain implementations, the adjustment vector includes distinct dimensions for baseline-relative trend weighting, cohort-relative scaling, and demographic calibration, each encoded using fixed-position vector segments or named fields within a model-conformant scoring artifact. These adjustment vectors are machine-readable and propagate through scoring workflows via inference result delivery interface 318, ensuring consistent downstream handling, auditability, and integration with competition rule logic configured within competition management system interface 302.

In some embodiments, handicap modeling engine 310 includes situation-aware transformation logic to ensure the adjustment vectors remain valid across multiple scoring contexts. For example, if the user performs a workout in a different modality than their baseline activity (e.g., trail run versus treadmill), the engine applies inter-modality scaling coefficients or context-aware offset transformations using compatibility matrices stored in item attribute datastore 332. This preserves interpretability and technical fairness in scenarios where metric distributions may vary systematically across modality types or device ecosystems. Each adjustment vector output is version-controlled and stored with lineage metadata to support reproducibility, rollback, and cross-model comparison.

Once the handicap adjustment vector has been generated, the system proceeds to compute a normalized score by applying configurable scoring logic that integrates the user's current metric values, individualized baselines, and the adjustment vector components. This step enables structured transformation of calibrated health metrics into context-sensitive, cohort-aware scores that are reproducible across activity types and device modalities. The normalized score reflects the user's relative performance in view of historical trends, population dynamics, and demographic calibration parameters, and serves as the output of the scoring pipeline for use in competitive ranking, feedback delivery, or personalized recommendations.

For example, in step 616, a normalized score is determined for the user by applying the handicap adjustment vector to current metric values in conjunction with individualized baselines and cohort-specific reference parameters. This computation is performed by inference system 110 using schema-conformant input vectors produced by feature engineering pipeline 306 and adjustment outputs from handicap modeling engine 310. The normalized score represents a calibrated, context-aware performance value computed from machine-executed transformations across input modalities, cohort contexts, and demographic conditions.

The scoring logic includes directionality-sensitive adjustments that account for whether improvement in a given metric corresponds to an increase or decrease in value. For example, a decrease in resting heart rate may be treated as an improvement, while an increase in step count may be similarly weighted. Each metric-specific adjustment is scaled by a corresponding component of the handicap adjustment vector, which includes per-metric weights reflecting demographic correction factors, cohort-relative deviation measures, and device calibration offsets. The scoring logic applies a weighted sum or composite scoring model (e.g., linear combination, multi-layer inference rule set, or ensemble regressor) to produce a final normalized score for the current evaluation interval.

In various embodiments, the normalized score is computed independently for each supported scoring context, such as modality-aligned activity segments or intra-device performance snapshots. For example, a user may receive a treadmill-specific score computed from metrics recorded during structured treadmill sessions, and a separate score reflecting overnight recovery quality based on HRV, REM duration, and sleep efficiency. Each of these context-specific scores is derived from the same underlying adjustment vector, but evaluated using context-restricted metric inputs and tailored scoring formulas. These scores may be combined into an aggregate competition score or used separately depending on platform configuration and competition rules.

The computed normalized score is tagged with a timestamp, scoring context label, version identifier for the applied adjustment model, and a provenance hash for the underlying metric inputs. This enables traceable, reproducible scoring output suitable for leaderboard display, fairness evaluation, or downstream analytics. In certain embodiments, the system stores both the final score and its component breakdown (e.g., contribution of each metric, adjustment factor, and baseline delta) to enable explainable inference and post-hoc validation of scoring fairness or accuracy. These outputs are transmitted to inference result delivery interface 318 for downstream routing, notification generation, or integration into user-facing feedback workflows.

In step 618, the system updates the challenge state based on the normalized score determined in step 616 and contextual parameters associated with the user's current competition participation. This update is performed by inference system 110 and coordination subsystems that manage the state of active challenges, track user progress, and determine eligibility for advancement, ranking, or reward distribution. The challenge state reflects a structured representation of the user's participation metrics within the configured competition format, including accumulated scores, completion status, ranking tier, or unlocked milestones.

The system retrieves the active challenge definition for the user from a competition rules repository, including challenge format (e.g., cumulative, head-to-head, threshold-based), scoring cadence (e.g., daily, weekly, rolling average), and eligibility conditions (e.g., minimum device support, cohort category, or baseline confidence score). Using this configuration, the system updates one or more challenge state records associated with the user by integrating the latest normalized score into the correct temporal bucket or scoring window. For example, in a 7-day cumulative challenge, the score may be added to the user's rolling total, while in a rank-based head-to-head challenge, the system may trigger a recomputation of leaderboards for the user's cohort and activity modality.

In certain embodiments, the challenge state update includes incrementing a milestone tracker, advancing the user's tier level, or flagging completion of a configured event sequence. These updates are governed by machine-executed rule sets defined in the challenge metadata and may include conditional transitions based on threshold attainment or relative ranking deltas. For example, if a user's cumulative score exceeds a pre-defined threshold for their cohort, the system may mark the challenge segment as complete and schedule a reward allocation process. If the user's rank changes in a competitive bracket, the system may log the change and trigger a notification pipeline.

Challenge state records are versioned and persisted with traceable metadata, including scoring inputs, adjustment model identifiers, and update timestamps. This enables auditability, rollback, and longitudinal progress tracking. In some embodiments, challenge states are synchronized across devices and interfaces via inference result delivery interface 318 to ensure consistent status display and eligibility enforcement. These machine-executed updates form the basis for user-facing feedback, leaderboard visualization, and competition-specific workflows in downstream components of the platform.

In step 620, the system provides the computed scoring results, challenge updates, and associated adjustment data to one or more output destinations based on the user's configured interface settings and system delivery logic. This output process is executed by inference result delivery interface 318, which transmits structured scoring artifacts to downstream systems, client applications, or storage components for presentation, synchronization, or archival. The result delivery is performed in a schema-conformant and machine-readable format, enabling consistent application across diverse interface types and ensuring reproducibility of the scoring outcome.

The results may include the user's latest normalized score, adjustment factor breakdowns, cohort-relative performance metrics, and updated challenge status. In certain embodiments, results are packaged as JSON or protocol buffer messages containing timestamped fields such as normalized_score, adjustment_vector, cohort_id, confidence_weight, and scoring_context. The scoring context may identify the activity type, competition phase, device metadata, and applicable scoring rule configuration used to compute the result. For example, the payload may indicate that a 14-day weighted baseline was applied for a treadmill-based session, using cohort calibration version 3.2 and adjustment model layer weights from model version 4.7.

In some embodiments, inference result delivery interface 318 includes transformation logic to format results for end-user presentation systems, such as converting numeric scores into visual progress bars, badge unlock events, or comparative cohort percentile ranks. The interface may also propagate result updates to user profile records, competition dashboards, or eligibility tracking modules. For multi-device consistency, scoring results may be broadcast to multiple endpoints (e.g., mobile app, wearable companion interface, web portal) using publish-subscribe mechanisms or push notification APIs governed by the user's platform settings.

Inference result delivery interface 318 supports traceable and version-controlled delivery of scoring outputs by embedding model version identifiers, input hash digests, and inference session metadata into each outbound record. This enables downstream systems to verify the origin, reproducibility, and timing of each score, and supports alignment with the active challenge state as updated in step 618. In certain embodiments, the results provided in step 620 include audit markers or performance traces for debugging, performance monitoring, or fairness analysis workflows. These machine-executed delivery operations complete the scoring lifecycle by transmitting context-aware, cohort-normalized, and adjustment-informed results to external systems or users in a consistent and verifiable format.

### Process for Generating Adjustment Vectors

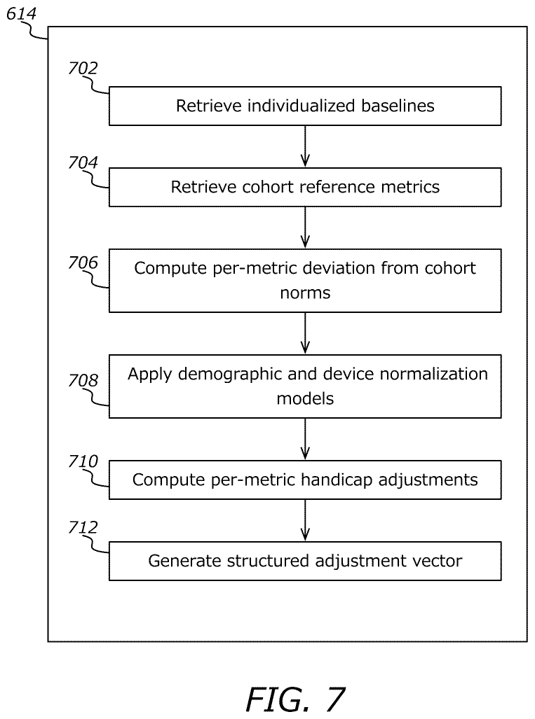

FIG. 7 illustrates an example process for generating a structured adjustment vector based on individualized baselines, cohort reference metrics, and demographic calibration parameters, in accordance with various embodiments. The method steps depicted in FIG. 7 may be implemented as processor-executable instructions stored in memory and executed by one or more computing components of the system architecture described in FIG. 1, including feature engineering pipeline 306, cohort modeling engine 308, and handicap modeling engine 310. In certain embodiments, the process may be invoked as part of the scoring workflow illustrated in FIG. 6 or as a standalone adjustment-generation routine triggered by new data ingestion, cohort reassignment, or model retraining events. The operations of FIG. 7 define the machine-executed transformations used to produce the schema-conformant adjustment vectors that drive normalized score computations across diverse activity modalities and population contexts.

In step 702, the system retrieves individualized baseline values for the user across a set of health metrics by accessing rolling-window baseline outputs generated by feature engineering pipeline 306. These baseline values serve as temporally weighted reference points for each tracked metric and reflect the user's recent physiological and behavioral patterns under consistent observation conditions. More specifically, feature engineering pipeline 306 accesses calibrated metric vectors stored in user preference datastore 330 and applies configurable recency-weighted smoothing functions to compute per-metric baselines, such as average resting heart rate, step count, HRV, or REM sleep duration over a rolling window (e.g., 14 days). Each baseline is stored as a time-indexed scalar tagged with modality context, device provenance, and metric identifier, enabling downstream compatibility with cohort-normalization workflows and adjustment factor generation logic. In certain embodiments, the retrieved baselines are filtered by scoring context (e.g., modality or challenge format) to support situation-aware evaluation in subsequent stages.

In step 702, individualized baseline values are retrieved for the user across a set of tracked health metrics. These values are computed by feature engineering pipeline 306 using recency-weighted rolling averages applied to calibrated metric data and serve as temporally weighted reference points for subsequent comparison to cohort-level expectations. More specifically, feature engineering pipeline 306 accesses calibrated metric vectors stored in user preference datastore 330 and applies configurable recency-weighted smoothing functions to compute per-metric baselines, such as average resting heart rate, step count, HRV, or REM sleep duration over a rolling window (e.g., 14 days). Each baseline is stored as a time-indexed scalar tagged with modality context, device provenance, and metric identifier, enabling downstream compatibility with cohort-normalization workflows and adjustment factor generation logic. In certain embodiments, the retrieved baselines are filtered by scoring context (e.g., modality or challenge format) to support situation-aware evaluation in subsequent stages.

In step 704, cohort reference metrics are retrieved for the assigned user cohort to establish population-normalized expectations for each tracked health metric. This retrieval operation is performed by cohort modeling engine 308, which accesses cohort-level aggregates stored in user preference datastore 330 and item attribute datastore 332. Each cohort reference metric is defined as a population baseline or expected distribution parameter (e.g., mean, median, standard deviation, or percentile curve) computed from historical calibrated metrics contributed by users assigned to the same cohort under matched modality and device configurations. For example, the average resting heart rate for a cohort of females aged 50-60 using wrist-based optical sensors may be 64.7 BPM, with a standard deviation of 3.2 BPM and a 90th percentile threshold at 69 BPM. These reference values are retrieved in a schema-conformant structure aligned to the internal scoring schema, with each metric tagged by cohort ID, measurement context, reference window, and aggregation method. In certain embodiments, the reference data includes modality-specific partitions to support situation-aware normalization, such that different cohort parameters may be used depending on whether the user's data was recorded during sleep tracking, aerobic activity, or strength training.

In step 706, the system computes a per-metric deviation between the user's individualized baseline values and the corresponding cohort reference metrics retrieved in step 704. This computation is performed by handicap modeling engine 310 using structured comparison logic that aligns each metric dimension from the user's baseline vector with the cohort's expected value under the same measurement context. For each health metric, the engine determines the signed delta (e.g., absolute or percentage deviation), percentile rank position, or z-score based on the distribution properties of the cohort reference. For example, if the user's baseline REM sleep duration is 92 minutes and the cohort's average is 78 minutes with a standard deviation of 10 minutes, the resulting z-score may be calculated as +1.4. The system stores these deviation values in an intermediate vector aligned with the user's scoring schema, annotating each entry with the metric ID, deviation type (e.g., mean-difference, percentile delta), and confidence weight based on the density and stability of the cohort reference data. In certain embodiments, outlier detection or smoothing filters may be applied to reduce sensitivity to anomalous readings or transient metric spikes prior to finalizing the deviation vector. This structured deviation output is used in subsequent modeling steps to produce calibrated handicap factors that reflect both individual progress and cohort-relative normalization targets.

In step 708, the system applies demographic and device normalization models to refine each element of the deviation vector computed in step 706. This process is performed by handicap modeling engine 310 using machine-executed model layers configured to ingest structured input vectors representing user-specific baselines, per-metric cohort deviations, and normalization factors derived from demographic attributes and device profile parameters. Each normalization model operates as a transformation function that modifies the raw deviation values to account for known distributional biases associated with user age, sex, or device characteristics. For example, if the user's step cadence is observed to deviate −15 steps/min from the cohort average, and the cohort contains a younger population using higher-fidelity foot pods, a demographic normalization layer may attenuate the deviation to −8 steps/min based on historical distribution adjustments. In some embodiments, linear regression coefficients, multiplicative scaling terms, or learned offset vectors are retrieved from model output log datastore 324 and applied per metric using vectorized operations. Device profile adjustments may further include sensor type correction (e.g., PPG vs. ECG), sampling resolution adjustments, or modality-specific error model compensation. Each correction is applied to a corresponding metric ID within the deviation vector, and annotated with the normalization source, confidence score, and model version used. The resulting corrected deviation vector enables downstream scoring logic to generate handicap factors that reflect not only cohort-relative variance, but also technical and physiological baselines appropriate to the user's profile.

In step 710, the system computes a handicap adjustment for each health metric by applying structured transformation logic to the corrected deviation values produced in step 708. This computation is executed by handicap modeling engine 310 using a configurable adjustment function that maps normalized deviation magnitudes to per-metric handicap weights. Each adjustment represents the system's quantification of how much a given metric should be adjusted to reflect an equitable scoring basis across differing user profiles. The adjustment function may be implemented as a piecewise scaling model, a bounded sigmoid transformation, or a lookup-based mapping derived from historical fairness calibration datasets. For example, a −12 BPM deviation in resting heart rate—corrected for age and device class—may map to a +0.15 handicap factor for the cardiovascular score dimension. Similarly, a positive deviation in REM sleep duration may yield a negative adjustment factor, depending on scoring directionality and the defined metric contribution. In certain embodiments, handicap modeling engine 310 retrieves scoring directionality metadata and weight mappings from item attribute datastore 332, ensuring that each adjustment is semantically aligned with the target scoring context. The per-metric handicap adjustments are computed in a consistent vectorized format, tagged with their originating deviation component, and labeled according to the metric schema in use. These structured adjustments serve as the core inputs for generating a normalized handicap vector in the next step.

In step 712, the system generates a structured adjustment vector by compiling the per-metric handicap adjustments computed in step 710 into a schema-conformant output artifact. This operation is executed by handicap modeling engine 310 and serves as the final transformation in the handicap generation sequence. The adjustment vector is formatted as a multi-dimensional scoring object in which each vector element corresponds to a normalized handicap factor for a specific health metric. In certain embodiments, each element includes the metric identifier, the computed adjustment value, and an optional confidence weight derived from the correction model in step 708. The adjustment vector may be implemented using a fixed-field schema (e.g., JSON schema or protocol buffer) with versioned field descriptors, ensuring compatibility with downstream scoring modules and reproducibility of the applied transformation. For example, the adjustment vector may contain entries such as {metric_id: ‘resting_hr’, value: 0.15, weight: 0.92} or {metric_id: ‘rem_sleep_pct’, value: −0.08, weight: 0.85}, depending on the scoring directionality and baseline deviation patterns. The structured adjustment vector is tagged with a model version identifier, cohort ID, scoring context, and timestamp to facilitate auditability and tracking across inference cycles. Once generated, the vector is stored in user preference datastore 330 and propagated to inference result delivery interface 318 for integration into normalized score computation as described in FIG. 6.

In accordance with various embodiments, FIG. 7 represents a set of sub-steps executed during step 614 of FIG. 6 for generating handicap factors using structured, machine-executed modeling logic. Each step in FIG. 7 corresponds to a discrete transformation applied by handicap modeling engine 310, beginning with individualized baseline inputs and cohort-derived reference metrics, and culminating in the generation of a structured adjustment vector. This adjustment vector forms the basis for downstream normalized score computations, enabling the system to apply demographic-aware, device-calibrated, and context-sensitive corrections to user health metrics in a reproducible and schema-conformant manner. As described, the operations performed in FIG. 7 reflect a layered modeling pipeline in which deviation detection, correction modeling, and adjustment factor synthesis are performed using traceable inputs and version-controlled scoring artifacts. These sub-operations may be implemented using statistical rule sets, trained machine learning models, or a hybrid of both, and are configured to support fairness, transparency, and reproducibility in scoring workflows across diverse user cohorts and device ecosystems.

### Hardware Architecture

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC), or on a network interface card.

Software/hardware hybrid implementations of at least some of the embodiments disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific embodiments, at least some of the features or functionalities of the various embodiments disclosed herein may be implemented on one or more general-purpose computers associated with one or more networks, such as for example an end-user computer system, a client computer, a network server or other server system, a mobile computing device (e.g., tablet computing device, mobile phone, smartphone, laptop, or other appropriate computing device), a consumer electronic device, a music player, or any other suitable electronic device, router, switch, or other suitable device, or any combination thereof. In at least some embodiments, at least some of the features or functionalities of the various embodiments disclosed herein may be implemented in one or more virtualized computing environments (e.g., network computing clouds, virtual machines hosted on one or more physical computing machines, or other appropriate virtual environments).

Any of the above-mentioned systems, modules, engines, interfaces, components, or the like may comprise hardware and/or software as described herein. For example, the systems described in association with health data acquisition system interface 304, feature engineering pipeline 306, cohort modeling engine 308, handicap modeling engine 310, training coordination module 312, drift detection module 316, inference result delivery interface 318, and model storage and version control system 320, as well as subcomponents thereof, may comprise computing hardware and/or software implementations. Furthermore, any of the above-mentioned systems, modules, engines, interfaces, components, or the like may include and/or interact with an application programming interface (API) for communicating with other systems, modules, engines, interfaces, or components for obtaining and/or providing data or instructions.

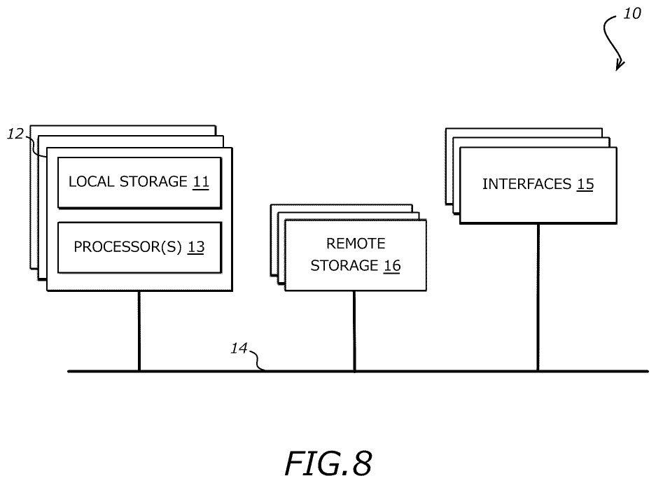

Referring now to FIG. 8, there is shown a block diagram depicting an exemplary computing device 10 suitable for implementing at least a portion of the features or functionalities disclosed herein. Computing device 10 may be, for example, any one of the computing machines listed in the previous paragraph, or indeed any other electronic device capable of executing software-or hardware-based instructions according to one or more programs stored in memory. Computing device 10 may be configured to communicate with a plurality of other computing devices, such as clients or servers, over communications networks such as a wide area network a metropolitan area network, a local area network, a wireless network, the Internet, or any other network, using known protocols for such communication, whether wireless or wired.

In one aspect, computing device 10 includes one or more central processing units (CPU) 12, one or more interfaces 15, and one or more busses 14 (such as a peripheral component interconnect (PCI) bus). When acting under the control of appropriate software or firmware, CPU 12 may be responsible for implementing specific functions associated with the functions of a specifically configured computing device or machine. For example, in at least one aspect, a computing device 10 may be configured or designed to function as a server system utilizing CPU 12, local memory 11 and/or remote memory 16, and interface(s) 15. In at least one aspect, CPU 12 may be caused to perform one or more of the different types of functions and/or operations under the control of software modules or components, which for example, may include an operating system and any appropriate applications software, drivers, and the like.

CPU 12 may include one or more processors 13 such as, for example, a processor from one of the Intel, ARM, Qualcomm, and AMD families of microprocessors. In some embodiments, processors 13 may include specially designed hardware such as application-specific integrated circuits (ASICs), electrically erasable programmable read-only memories (EEPROMs), field-programmable gate arrays (FPGAs), and so forth, for controlling operations of computing device 10. In a particular aspect, a local memory 11 (such as non-volatile random-access memory (RAM) and/or read-only memory (ROM), including for example one or more levels of cached memory) may also form part of CPU 12. However, there are many different ways in which memory may be coupled to system 10. Memory 11 may be used for a variety of purposes such as, for example, caching and/or storing data, programming instructions, and the like. It should be further appreciated that CPU 12 may be one of a variety of system-on-a-chip (SOC) type hardware that may include additional hardware such as memory or graphics processing chips, such as a QUALCOMM SNAPDRAGON™ or SAMSUNG EXYNOS™ CPU as are becoming increasingly common in the art, such as for use in mobile devices or integrated devices.

As used herein, the term “processor” is not limited merely to those integrated circuits referred to in the art as a processor, a mobile processor, or a microprocessor, but broadly refers to a microcontroller, a microcomputer, a programmable logic controller, an application-specific integrated circuit, and any other programmable circuit.

In one aspect, interfaces 15 are provided as network interface cards (NICs). Generally, NICs control the sending and receiving of data packets over a computer network; other types of interfaces 15 may for example support other peripherals used with computing device 10. Among the interfaces that may be provided are Ethernet interfaces, frame relay interfaces, cable interfaces, DSL interfaces, token ring interfaces, graphics interfaces, and the like. In addition, various types of interfaces may be provided such as, for example, universal serial bus (USB), Serial, Ethernet, FIREWIRE™, THUNDERBOLT™, PCI, parallel, radio frequency (RF), BLUETOOTH™, near-field communications (e.g., using near-field magnetics), 802.11 (WiFi), frame relay, TCP/IP, ISDN, fast Ethernet interfaces, Gigabit Ethernet interfaces, Serial ATA (SATA) or external SATA (ESATA) interfaces, high-definition multimedia interface (HDMI), digital visual interface (DVI), analog or digital audio interfaces, asynchronous transfer mode (ATM) interfaces, high-speed serial interface (HSSI) interfaces, Point of Sale (POS) interfaces, fiber data distributed interfaces (FDDIs), and the like. Generally, such interfaces 15 may include physical ports appropriate for communication with appropriate media. In some cases, they may also include an independent processor (such as a dedicated audio or video processor, as is common in the art for high-fidelity A/V hardware interfaces) and, in some instances, volatile and/or non-volatile memory (e.g., RAM).

Although the system shown in FIG. 8 illustrates one specific architecture for a computing device 10 for implementing one or more of the embodiments described herein, it is by no means the only device architecture on which at least a portion of the features and techniques described herein may be implemented. For example, architectures having one or any number of processors 13 may be used, and such processors 13 may be present in a single device or distributed among any number of devices. In one aspect, single processor 13 handles communications as well as routing computations, while in other embodiments a separate dedicated communications processor may be provided. In various embodiments, different types of features or functionalities may be implemented in a system according to the aspect that includes a client device (such as a tablet device or smartphone running client software) and server systems (such as a server system described in more detail below).

Regardless of network device configuration, the system of an aspect may employ one or more memories or memory modules (such as, for example, remote memory block 16 and local memory 11) configured to store data, program instructions for the general-purpose network operations, or other information relating to the functionality of the embodiments described herein (or any combinations of the above). Program instructions may control execution of or comprise an operating system and/or one or more applications, for example. Memory 16 or memories 11, 16 may also be configured to store data structures, configuration data, encryption data, historical system operations information, or any other specific or generic non-program information described herein.

Because such information and program instructions may be employed to implement one or more systems or methods described herein, at least some network device embodiments may include nontransitory machine-readable storage media, which, for example, may be configured or designed to store program instructions, state information, and the like for performing various operations described herein. Examples of such nontransitory machine-readable storage media include, but are not limited to, magnetic media such as hard disks, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device), or they may be removable such as swappable flash memory modules (such as “thumb drives” or other removable media designed for rapidly exchanging physical storage devices), “hot-swappable” hard disk drives or solid state drives, removable optical storage discs, or other such removable media, and that such integral and removable storage media may be utilized interchangeably. Examples of program instructions include both object code, such as may be produced by a compiler, machine code, such as may be produced by an assembler or a linker, byte code, such as may be generated by for example a JAVA™ compiler and may be executed using a Java virtual machine or equivalent, or files containing higher level code that may be executed by the computer using an interpreter (for example, scripts written in Python, Perl, Ruby, Groovy, or any other scripting language).

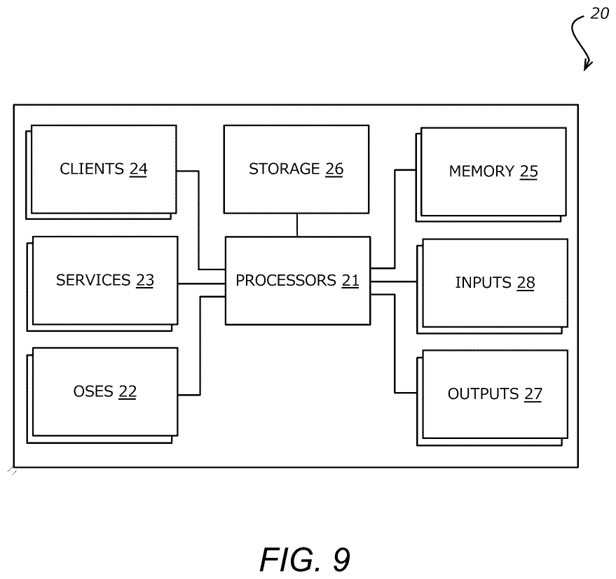

In some embodiments, systems may be implemented on a standalone computing system. Referring now to FIG. 9, there is shown a block diagram depicting a typical exemplary architecture of one or more embodiments or components thereof on a standalone computing system. Computing device 20 includes processors 21 that may run software that carry out one or more functions or applications of embodiments, such as for example a client application. Processors 21 may carry out computing instructions under control of an operating system 22 such as, for example, a version of MICROSOFT WINDOWS™ operating system, APPLE macOS™ or iOS™ operating systems, some variety of the Linux operating system, ANDROID™ operating system, or the like. In many cases, one or more shared services 23 may be operable in system 20, and may be useful for providing common services to client applications. Services 23 may for example be WINDOWS™ services, user-space common services in a Linux environment, or any other type of common service architecture used with operating system 21. Input devices 28 may be of any type suitable for receiving user input, including for example a keyboard, touchscreen, microphone (for example, for voice input), mouse, touchpad, trackball, or any combination thereof. Output devices 27 may be of any type suitable for providing output to one or more users, whether remote or local to system 20, and may include for example one or more screens for visual output, speakers, printers, or any combination thereof. Memory 25 may be random-access memory having any structure and architecture known in the art, for use by processors 21, for example to run software. Storage devices 26 may be any magnetic, optical, mechanical, memristor, or electrical storage device for storage of data in digital form (such as those described above, referring to FIG. 8). Examples of storage devices 26 include flash memory, magnetic hard drive, CD-ROM, and/or the like.

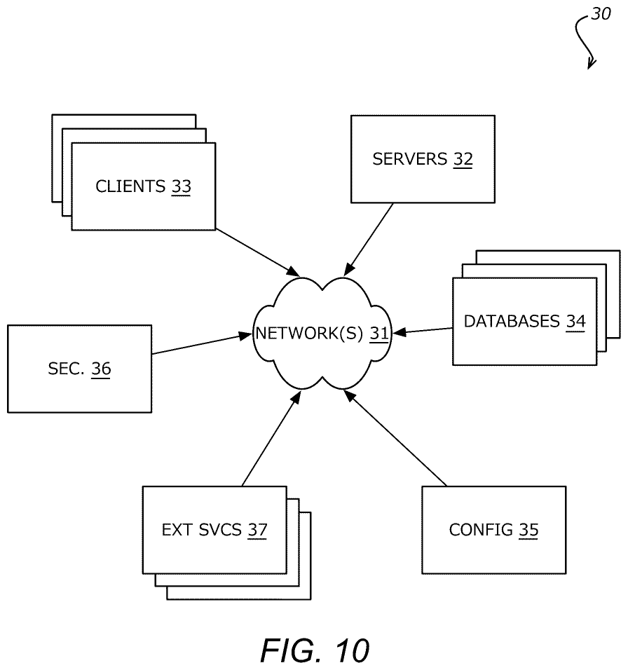

In some embodiments, systems may be implemented on a distributed computing network, such as one having any number of clients and/or servers. Referring now to FIG. 10, there is shown a block diagram depicting an exemplary architecture 30 for implementing at least a portion of a system according to one aspect on a distributed computing network. According to the aspect, any number of clients 33 may be provided. Each client 33 may run software for implementing client-side portions of a system; clients may comprise a system 20 such as that illustrated in FIG. 9. In addition, any number of servers 32 may be provided for handling requests received from one or more clients 33. Clients 33 and servers 32 may communicate with one another via one or more electronic networks 31, which may be in various embodiments any of the Internet, a wide area network, a mobile telephony network (such as CDMA or GSM cellular networks), a wireless network (such as WiFi, WiMAX, LTE, and so forth), or a local area network (or indeed any network topology known in the art; the aspect does not prefer any one network topology over any other). Networks 31 may be implemented using any known network protocols, including for example wired and/or wireless protocols.

In addition, in some embodiments, servers 32 may call external services 37 when needed to obtain additional information, or to refer to additional data concerning a particular call. Communications with external services 37 may take place, for example, via one or more networks 31. In various embodiments, external services 37 may comprise web-enabled services or functionality related to or installed on the hardware device itself. For example, in one aspect where client applications are implemented on a smartphone or other electronic device, client applications may obtain information stored in a server system 32 in the cloud or on an external service 37 deployed on one or more of a particular enterprise's or user's premises.

In some embodiments, clients 33 or servers 32 (or both) may make use of one or more specialized services or appliances that may be deployed locally or remotely across one or more networks 31. For example, one or more databases 34 may be used or referred to by one or more embodiments. It should be understood by one having ordinary skill in the art that databases 34 may be arranged in a wide variety of architectures and using a wide variety of data access and manipulation means. For example, in various embodiments one or more databases 34 may comprise a relational database system using a structured query language (SQL), while others may comprise an alternative data storage technology such as those referred to in the art as “NoSQL” (for example, HADOOP CASSANDRA™, GOOGLE BIGTABLE™, and so forth). In some embodiments, variant database architectures such as column-oriented databases, in-memory databases, clustered databases, distributed databases, or even flat file data repositories may be used according to the aspect. It will be appreciated by one having ordinary skill in the art that any combination of known or future database technologies may be used as appropriate, unless a specific database technology or a specific arrangement of components is specified for a particular aspect described herein. Moreover, it should be appreciated that the term “database” as used herein may refer to a physical database machine, a cluster of machines acting as a single database system, or a logical database within an overall database management system. Unless a specific meaning is specified for a given use of the term “database”, it should be construed to mean any of these senses of the word, all of which are understood as a plain meaning of the term “database” by those having ordinary skill in the art.

Similarly, some embodiments may make use of one or more security systems 36 and configuration systems 35. Security and configuration management are common information technology (IT) and web functions, and some amount of each are generally associated with any IT or web systems. It should be understood by one having ordinary skill in the art that any configuration or security subsystems known in the art now or in the future may be used in conjunction with embodiments without limitation, unless a specific security 36 or configuration system 35 or approach is specifically required by the description of any specific aspect.

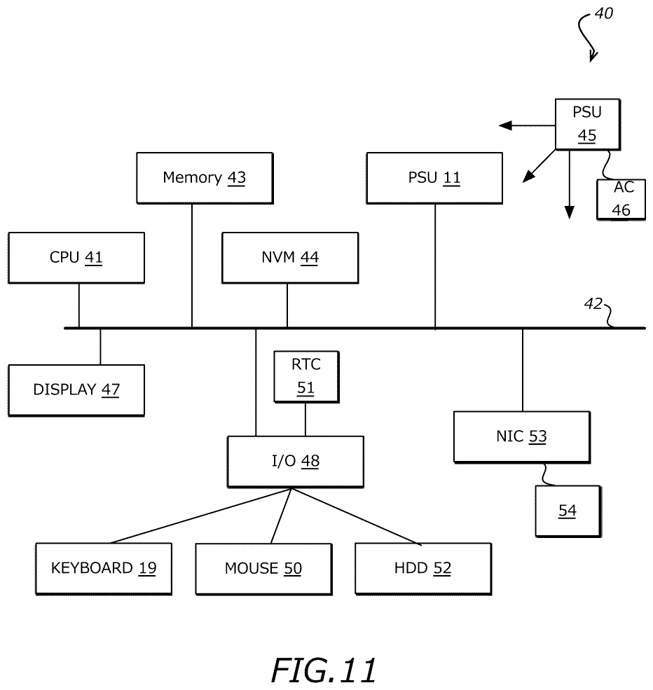

FIG. 11 shows an exemplary overview of a computer system 40 as may be used in any of the various locations throughout the system. It is exemplary of any computer that may execute code to process data. Various modifications and changes may be made to computer system 40 without departing from the broader scope of the system and method disclosed herein. Central processor unit (CPU) 41 is connected to bus 42, to which bus is also connected memory 43, nonvolatile memory 44, display 47, input/output (I/O) unit 48, and network interface card (NIC) 53. I/O unit 48 may, typically, be connected to keyboard 49, pointing device 50, hard disk 52, and real-time clock 51. NIC 53 connects to network 54, which may be the Internet or a local network, which local network may or may not have connections to the Internet. Also shown as part of system 40 is power supply unit 45 connected, in this example, to a main alternating current (AC) supply 46. Not shown are batteries that could be present, and many other devices and modifications that are well known but are not applicable to the specific novel functions of the current system and method disclosed herein. It should be appreciated that some or all components illustrated may be combined, such as in various integrated applications, for example Qualcomm or Samsung system-on-a-chip (SOC) devices, or whenever it may be appropriate to combine multiple capabilities or functions into a single hardware device (for instance, in mobile devices such as smartphones, video game consoles, in-vehicle computer systems such as navigation or multimedia systems in automobiles, or other integrated hardware devices).

In various embodiments, functionality for implementing systems or methods of various embodiments may be distributed among any number of client and/or server components. For example, various software modules may be implemented for performing various functions in connection with the system of any particular aspect, and such modules may be variously implemented to run on server and/or client components.

The skilled person will be aware of a range of possible modifications of the various embodiments described above. Accordingly, the present invention is defined by the claims and their equivalents.

### Additional Considerations

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for facilitating database queries through the disclosed principles herein. Thus, while particular embodiments and applications have been illustrated and described, it is to be understood that the disclosed embodiments are not limited to the precise construction and components disclosed herein. Various apparent modifications, changes and variations may be made in the arrangement, operation and details of the method and apparatus disclosed herein without departing from the spirit and scope defined in the appended claims.

What is claimed is:

1. A computing system for generating handicap-adjusted health metric scores, the computing system comprising:
a computing device processor; and
a memory storing instructions that, when executed by the computing device processor, cause the computing system to:
obtain multi-source health data from a plurality of health tracking sources, the multi-source health data comprising unstructured health metric data associated with a user, the unstructured health metric data including heterogeneous metric values collected over time from disparate device types or activity modalities;
normalize the unstructured health metric data to produce a set of standardized metric values;
calibrate the standardized metric values using device profile data associated with each health tracking source, the device profile data comprising known measurement deviations or accuracy parameters for each device;
generate, for each metric, a baseline value for the user based on a rolling time window applied to calibrated metric values, the rolling time window defined by a configurable recency interval;
segment a population of users into a plurality of population cohorts based on demographic data and device metadata associated with the users;
assign the user to a selected population cohort of the plurality of population cohorts based on the demographic data and device metadata associated with the user;
retrieve one or more cohort reference parameters associated with the selected population cohort, each cohort reference parameter corresponding to a respective health metric;
compare, for each health metric, the baseline value for the user to corresponding cohort reference parameter to generate a deviation value;
apply one or more correction factors to each deviation value, the one or more correction factors derived from the demographic data and device metadata associated with the user;
generate a handicap adjustment vector comprising per-metric adjustment values based on corrected deviation values;
compute a normalized score for the user using the handicap adjustment vector and current metric values associated with the user; and
output the normalized score for presentation via a user interface associated with a health competition platform.

2. The computing system of claim 1, wherein the heterogeneous metric values comprise at least one of: heart rate, resting heart rate, steps, calories, distance, heart rate variability, sleep efficiency, or perceived exertion, and are collected from activity modalities comprising at least one of: walking, running, cycling, swimming, resistance training, mindfulness sessions, or sleep tracking.

3. The computing system of claim 1, wherein the instructions, when executed by the computing device processor to normalize the unstructured health metric data, further enables the computing system to:
convert units of measure, aligning temporal sampling rates, and mapping data fields from source-specific schemas to a standardized schema.

4. The computing system of claim 1, wherein the instructions, when executed by the computing device processor to calibrate the standardized metric values, further enables the computing system to:
apply device-specific correction coefficients stored in a device profile datastore, the device-specific correction coefficients derived from empirical accuracy ratings or known measurement bias associated with each device model.

5. The computing system of claim 1, wherein the baseline value is computed using a weighted moving average applied to calibrated metric values over the rolling time window, the weighted moving average decreasing with recency to bias more recent data.

6. The computing system of claim 1, wherein the instructions, when executed by the computing device processor to segment the population of users into cohorts, further enables the computing system to:
apply an unsupervised clustering model to user demographic features and device metadata to group users with similar performance characteristics.

7. The computing system of claim 1, wherein assigning the user to the selected population cohort comprises comparing a user's demographic features and device metadata to cluster centroids.

8. The computing system of claim 1, wherein the instructions, when executed by the computing device processor to compare baselines value to cohort reference parameters, further enables the computing system to:
compute a deviation ratio for each metric, the deviation ratio representing a normalized difference between user-specific and population-based expectations.

9. The computing system of claim 1, wherein the instructions, when executed by the computing device processor, further enables the computing system to:
detect a drift in at least one cohort reference parameter or baseline value by monitoring scoring stability metrics over time;
in response to detecting drift, triggering a retraining task for a population modeling system;
retrain the population modeling system using updated health metric data vectors;
validate a retrained model using cohort accuracy thresholds; and
deploy the retrained model when validation criteria are satisfied.

10. The computing system of claim 1, wherein the instructions, when executed by the computing device processor to compute the normalized score, further enables the computing system to:
apply a directionality adjustment to each metric based on whether improvement is represented by an increase or decrease in metric value.

11. A computer-implemented method for generating handicap-adjusted health metric scores, the computer-implemented method comprising:
obtaining multi-source health data from a plurality of health tracking sources, the multi-source health data comprising unstructured health metric data associated with a user, the unstructured health metric data including heterogeneous metric values collected over time from disparate device types or activity modalities;
normalizing the unstructured health metric data to produce a set of standardized metric values;
calibrating the standardized metric values using device profile data associated with each health tracking source, the device profile data comprising known measurement deviations or accuracy parameters for each device;
generating, for each metric, a baseline value for the user based on a rolling time window applied to calibrated metric values, the rolling time window defined by a configurable recency interval;
segmenting a population of users into a plurality of population cohorts based on demographic data and device metadata associated with the users;
assigning the user to a selected population cohort of the plurality of population cohorts based on the demographic data and device metadata associated with the user;
retrieving one or more cohort reference parameters associated with the selected population cohort, each cohort reference parameter corresponding to a respective health metric;
comparing, for each health metric, the baseline value for the user to a corresponding cohort reference parameter to generate a deviation value;
applying one or more correction factors to each deviation value, the one or more correction factors derived from the demographic data and device metadata associated with the user;
generating a handicap adjustment vector comprising per-metric adjustment values based on corrected deviation values;
computing a normalized score for the user using the handicap adjustment vector and current metric values associated with the user; and
outputting the normalized score for presentation via a user interface associated with a health competition platform.

12. The computer-implemented method of claim 11, wherein computing the normalized score further comprises:
applying population-level normalization factors derived from cohort reference statistics, the population-level normalization factors computed by comparing aggregated baseline distributions across multiple user cohorts to ensure scoring consistency within overlapping demographic or device categories.

13. The computer-implemented method of claim 11, further comprising:
applying cross-modality translation logic configured to map metric values from a first activity type to a normalized representation of a second activity type using modality-specific scaling parameters stored in a modality translation datastore, thereby enabling scoring equivalency across distinct activity modalities.

14. The computer-implemented method of claim 11, wherein computing the normalized score further comprises:
selecting a scoring pathway based on contextual tags associated with user data, the contextual tags comprising at least one of: device identity, competition format, team participation status, or environmental condition, a selected scoring pathway determining which calibration, normalization, and weighting rules are applied.

15. The computer-implemented of claim 11, wherein computing the normalized score comprises applying a directionality adjustment to each metric based on whether improvement is represented by an increase or decrease in metric value.

16. The computer-implemented method of claim 11, further comprising:
detecting a drift in at least one cohort reference parameter or baseline value by monitoring scoring stability metrics over time;
in response to detecting drift, triggering a retraining task for a population modeling system;
retraining the population modeling system using updated health metric data vectors;
validating a retrained model using cohort accuracy thresholds; and
deploying the retrained model when validation criteria are satisfied.

17. The computer-implemented method of claim 11, wherein generating the handicap adjustment vector comprises, for each metric, computing a weighted combination of:
a ratio between a current metric value and a corresponding baseline value for the user;
a demographic correction factor associated with the user;
a device correction factor associated with respective health tracking source;
a behavioral adjustment factor; and
a mental health adjustment factor.

18. A non-transitory computer-readable storage medium storing instructions that, when executed by one or more processors, cause a computing system to:
obtain multi-source health data from a plurality of health tracking sources, the multi-source health data comprising unstructured health metric data associated with a user, the unstructured health metric data including heterogeneous metric values collected over time from disparate device types or activity modalities;
normalize the unstructured health metric data to produce a set of standardized metric values;
calibrate the standardized metric values using device profile data associated with each health tracking source, the device profile data comprising known measurement deviations or accuracy parameters for each device;
generate, for each metric, a baseline value for the user based on a rolling time window applied to calibrated metric values, the rolling time window defined by a configurable recency interval;
segment a population of users into a plurality of population cohorts based on demographic data and device metadata associated with the users;
assign the user to a selected population cohort of the plurality of population cohorts based on the demographic data and device metadata associated with the user;
retrieve one or more cohort reference parameters associated with the selected population cohort, each cohort reference parameter corresponding to a respective health metric;
compare, for each health metric, the baseline value for the user to corresponding cohort reference parameter to generate a deviation value;
apply one or more correction factors to each deviation value, the one or more correction factors derived from the demographic data and device metadata associated with the user;
generate a handicap adjustment vector comprising per-metric adjustment values based on corrected deviation values;
compute a normalized score for the user using the handicap adjustment vector and current metric values associated with the user; and
output the normalized score for presentation via a user interface associated with a health competition platform.

19. The non-transitory computer-readable storage medium of claim 18, wherein computing the normalized score comprises applying: (i) a user-specific baseline deviation factor, (ii) a cohort-level percentile normalization factor, and (iii) a population-level scaling parameter.

20. The non-transitory computer-readable storage medium of claim 18, wherein the instructions, when executed by the one or more processors to generate the handicap adjustment vector, further enables the computing system to:
determine, for each metric, a weighted combination of a ratio between a current metric value and a baseline value together with demographic, device, behavioral, and mental-health adjustment factors.
